(19) United States
(12) Patent Application Publication
(10) Pub. No.: US 2010/0240616 A1
(43) Pub. Date: Sep. 23, 2010

(54) NOVEL LIPID COMPOUNDS

(76) Inventors: Anne Kristin Holmeide, Oslo (NO); Jenny Rosman, Marstrand (SE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/447,092

(22) PCT Filed: Nov. 1, 2007
(86) PCT No.: PCT/IB07/04588

§ 371 Date: Jul. 27, 2009

Related U.S. Application Data
(60) Provisional application No. 60/855,733, filed on Nov. 1, 2006.
(60) Provisional application No. 60/856,267, filed on Nov. 3, 2006.
(60) Provisional application No. 60/856,268, filed on Nov. 3, 2006.
(60) Provisional application No. 60/856,269, filed on Nov. 3, 2006.

(30) Foreign Application Priority Data
Nov. 1, 2006 (SE) 0602310-5
Nov. 3, 2006 (SE) 0602352-7

(51) Int. Cl. A61K 31/661; C07C 69/347; A61K 31/22; C07C 67/44; C07F 9/113; C07C 305/14; A61K 31/255; C07C 57/03; A61K 31/232; C07C 67/08; C07C 69/67; A61K 31/225; C07C 69/96; C07C 68/06; A61P 29/00; A61P 3/10; A61P 3/06; A61P 3/04; A61P 1/16

(52) U.S. Cl. 514/134; 560/261; 514/546; 560/238; 558/213; 558/115; 558/36; 514/517; 554/224; 514/549; 554/170; 560/129; 560/180; 514/547; 558/277

(57) ABSTRACT

Omega-3 lipid compounds of the general formula (I):

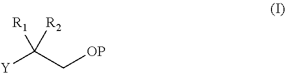

wherein R₁ and R₂ are the same or different and are chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
P represents a hydrogen atom, (Formula II) wherein P₂, P₃, and P₄ are chosen from a hydrogen atom, an alkyl group, and a C₁₄-C₂₂ alkenyl group, wherein the alkyl and alkenyl groups are optionally substituted with a hydroxy group, (Formula III), (Formula IV), or (Formula V); and
Y is a C₁₄-C₂₂ alkenyl group with at least one double bond, having E and/or Z configuration; or any pharmaceutically acceptable complex, solvate, salt or pro-drug thereof, with the proviso that R₁ and R₂ are not simultaneously a hydrogen atom. Also disclosed are pharmaceutical compositions and lipid compositions comprising such compounds, and to such compounds for use as medicaments, in particular for the treatment of cardiovascular and metabolic diseases.

### FIELD OF THE INVENTION

The present invention relates to omega-3 lipid compounds of the general formula (I):

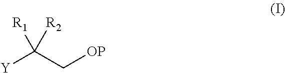

wherein R₁, R₂, P, and Y are herein defined.

The invention also relates to pharmaceutical compositions and lipid compositions comprising such compounds, and to such compounds for use as medicaments, in particular for the treatment of cardiovascular and metabolic diseases.

### BACKGROUND OF THE INVENTION

Dietary polyunsaturated fatty acids (PUFAs) have effects on diverse physiological processes impacting normal health and chronic diseases, such as the regulation of plasma lipid levels, cardiovascular and immune functions, insulin action, and neuronal development and visual function. Ingestion of PUFAs (generally in ester form, e.g. glycerides and phospholipids) will lead to their distribution to virtually every cell in the body with effects on membrane composition and function, eicosanoid synthesis, cellular signaling, and regulation of gene expression. Variations in distribution of different fatty acids/lipids to different tissues in addition to cell specific lipid metabolism, as well as the expression of fatty acid-regulated transcription factors, is likely to play an important role in determining how cells respond to changes in PUFA composition. (Benatti, P. Et al, J. Am. Coll. Nutr. 2004, 23, 281). PUFAs or their metabolites have been shown to modulate gene transcription by interacting with several nuclear receptors. These are the peroxisome proliferators-activated receptors (PPARs), the hepatic nuclear receptor (HNF-4), liver X receptor (LXR), and the 9-cis retinoic acid receptor (retinoic X receptor, RXR). Treatment with PUFAs can also regulate the abundance of many transcriptional factors in the nucleus, including SREBP, NFkB, c/EBPβ, and HIF-1α. These effects are not due to direct binding of the fatty acid to the transcription factor, but rather involve mechanisms that affect the nuclear content of the transcription factors. The regulation of gene transcription by PUFAs has profound effects on cell and tissue metabolism and offers a credible explanation for the involvement of nutrient-gene interactions in the initiation and prevention or amelioration of diseases such as obesity, diabetes, cardiovascular disorders, immune-inflammatory diseases and cancers (Wahle, J., et al, Proceedings of the Nutrition Society, 2003, 349). Fish oils rich in the ω-3 polyunsaturated fatty acids eicosapentaenoic acid (EPA) and docosahexaenoic acid (DHA), have been shown to reduce the risk of cardiovascular diseases partly by reduction of blood triglyceride concentration. This favorable effect mainly results from the combined effects of inhibition of lipogenesis by decrease of SPEBP-1 and stimulation of fatty acid oxidation by activation of PPAR-α in the liver.

Due to their limited stability in vivo and their lack of biological specificity, PUFAs have not achieved widespread use as therapeutic agents. Chemical modifications of the n-3 polyunsaturated fatty acids have been performed by several research groups in order to change or increase their metabolic effects.

For example, the hypolipidemic effects of EPA was potentiated by introducing methyl or ethyl in α-position of EPA ethyl ester (EE). (Vaagenes, et al, Biochemical Pharmacology, 1999, 58, 1133). Those compounds also reduced plasma free fatty acids, while the EPA EE compound had no effect.

In a recent work published by L. Larsen (Larsen, L. et al, Lipids, 2005, 40, 49), the authors show that the α-methyl derivatives of EPA and DHA increased the activation of the nuclear receptor PPARα and thereby the expression of L-FABP when compared to EPA/DHA. The authors suggest that delayed catabolism of these α-methyl PUFAs contribute to their increased effects.

### SUMMARY OF THE INVENTION

One object of the present invention is to provide omega-3 lipid compounds having pharmaceutical activity.

This object is achieved by an omega-3 lipid compound of formula (I):

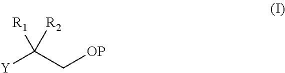

wherein

R₁ and R₂ are the same or different and are chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

P represents a hydrogen atom,

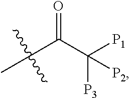

wherein P₁, P₂, and P₃ are chosen from a hydrogen atom, an alkyl group, and a C₁₄-C₂₂ alkenyl group, wherein the alkyl and alkenyl groups are optionally substituted with a hydroxy group,

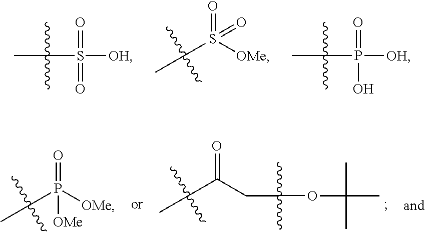

Y is a C₁₄-C₂₂ alkenyl group with at least one double bond, having E and/or Z configuration;

or any pharmaceutically acceptable complex, solvate, salt or pro-drug thereof, with the proviso that R₁ and R₂ are not simultaneously a hydrogen atom.

In particular, the present invention relates to omega-3 lipid compounds of formula (I) wherein:
Y is a C₁₆-C₂₂ alkenyl with 2-6 double bonds;
Y is a C₁₆-C₂₀ alkenyl with 2-6 methylene interrupted double bonds in Z configuration;
Y is a C₂₀ alkenyl with 6 methylene interrupted double bonds in Z configuration;
Y is a C₂₀ alkenyl with 5 methylene interrupted double bonds in Z configuration
Y is a C₁₆-C₂₀ alkenyl with 3-5 double bonds;
Y is a C₁₆-C₇₀ alkenyl with 3-5 methylene interrupted double bonds in Z configuration;
Y is a C₁₈ alkenyl with 5 methylene interrupted double bonds in Z configuration;
Y is a C₁₆ alkenyl with 3 double bonds in Z-configuration; or
Y is a C₁₆ alkenyl with 3 methylene interrupted double bonds in Z configuration.

More precisely, the present invention relates to an omega-3 lipid compound selected from the group consisting of:
(all-Z)-4,7,10,13,16,19-docosahexaen-1-ol,
(all-Z)-5,8,11,14,17-eicosapentaen-1-ol,
(all-Z)-9,12,15-octadecatrien-1-ol,
(all-Z)-7,10,13,16,19-docosapentaen-1-ol,
(all-Z)-11,14,17-eicosatrien-1-ol,
(4E,8Z,11Z,14Z,17Z)-eicosapentaen-1-ol,
(5E,8Z,11Z,14Z,17Z)-eicosapentaen-1-ol, and
(4E,7Z,10Z,13Z,16Z,19Z)-docosahexaen-1-ol.
   or any pharmaceutically acceptable complex, solvate, salt, or pro-drug thereof,
   wherein said omega-3 lipid compound is substituted at carbon 2, counted from the hydroxyl functional group, with at least one substituent chosen from:
   a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
   with the provisos that:
   R₁ and R₂ are not simultaneously a hydrogen atom.

In exemplary embodiments of the invention, an omega-3 lipid compound is chosen from:

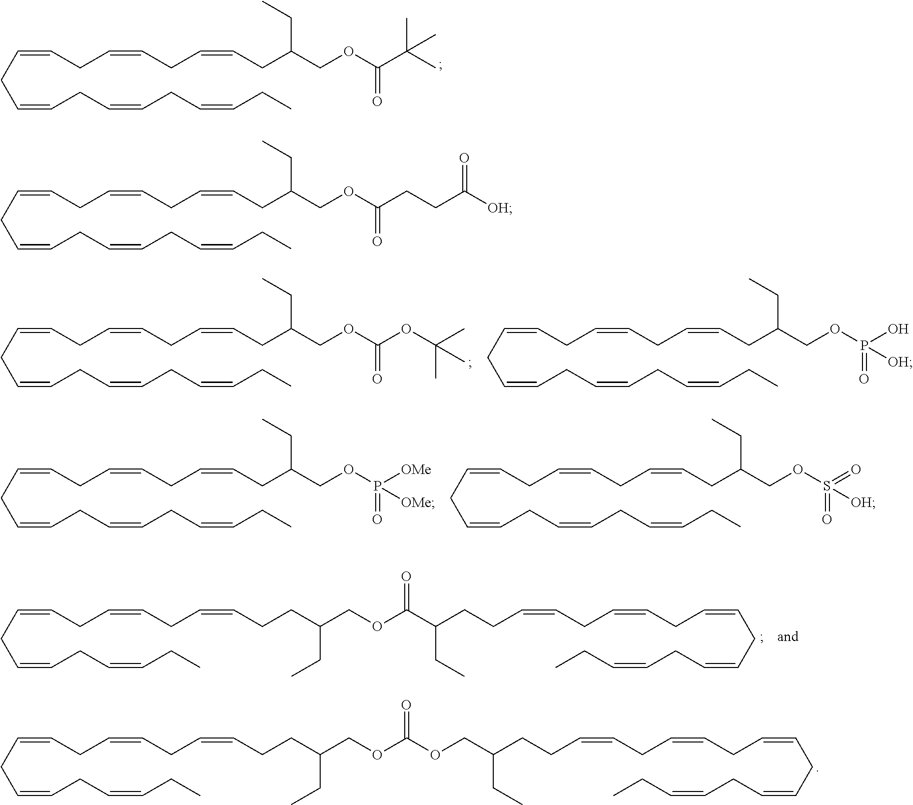

In a compound according to the invention, said alkyl group may be chosen from methyl, ethyl, n-propyl, isopropyl, n-butyl, iso-butyl, sec-butyl, and n-hexyl; said halogen atom may be fluorine; said alkoxy group may be chosen from methoxy, ethoxy, propoxy, isopropoxy, sec-butoxy, phenoxy, benzyloxy, OCH₂CF₃, and OCH₂CH₂OCH₃; said alkenyl group may be chosen from allyl, 2-butenyl, and 3-hexenyl; said alkynyl group may be chosen from propargyl, 2-butynyl, and 3-hexynyl; said aryl group may be chosen from a benzyl group, and a substituted benzyl group; said alkylthio group may be chosen from methylthio, ethylthio, isopropylthio, and phenylthio; said alkoxycarbonyl group may be chosen from methoxycarbonyl, ethoxycarbonyl, propoxycarbonyl, and butoxycarbonyl; said alkylsulfinyl group may be chosen from methanesulfinyl, ethanesulfinyl, and isopropanesulfinyl; said alkylsulfonyl group may be chosen from methanesulfonyl, ethanesulfonyl, and isopropanesulfonyl; and said alkylamino group may be chosen from methylamino, dimethylamino, ethylamino, and diethylamino.

In particular, R₁ and R₂ may be chosen from a hydrogen atom; an alkyl group, e.g. a C₁-C₇ alkyl group; an alkoxy group, e.g. a C₁-C₇ alkoxy group; an alkylthio group, e.g. a C₁-C₇ alkylthio group; an amino group, an alkylamino group, e.g. a C₁-C₇ alkylamino, an alkoxycarbonyl group, e.g. a C₁-C₇ alkoxycarbonyl group, and a carboxy group.

For example, said C₁-C₇ alkyl group may be methyl, ethyl, or propyl; said C₁-C₇ alkoxy group may be methoxy, ethoxy or propoxy; said C₁-C₇ alkylthio group may be methylthio, ethylthio, or propylthio; said C₁-C₇ alkylamino group may be ethylamino or diethylamino.

According to the present invention, P represents a hydrogen atom, or

P represents;

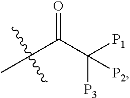

wherein P₁, P₂, and P₃ are chosen from a hydrogen atom, an alkyl group, and a C₁₄-C₂₂ alkenyl group, wherein the alkyl and alkenyl groups are optionally substituted with a hydroxy group, or

P represents;

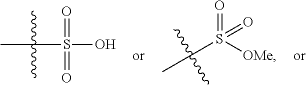

P represents;

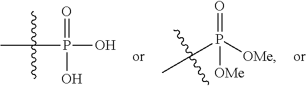

P represents;

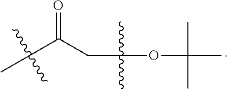

Examples of compounds according to the invention are those in which P is a hydrogen, and Y is a C₂₀ alkenyl with 6 methylene interrupted double bonds, located in Z configuration, wherein:

one of R₁ and R₂ is methyl and the other one is a hydrogen atom;

one of R₁ and R₂ is ethyl and the other one is a hydrogen atom;

one of R₁ and R₂ is propyl and the other one is a hydrogen atom;

one of R₁ and R₂ is methoxy and the other one is a hydrogen atom;

one of R₁ and R₂ is ethoxy and the other one is a hydrogen atom;

one of R₁ and R₂ is propoxy and the other one is a hydrogen atom;

one of R₁ and R₂ is thiomethyl and the other one is a hydrogen atom;

one of R₁ and R₂ is thioethyl and the other one is a hydrogen atom;

one of R₁ and R₂ is thiopropyl and the other one is a hydrogen atom;

one of R₁ and R₂ is ethylamino and the other one is a hydrogen atom;

one of R₁ and R₂ is benzyl and the other one is a hydrogen atom;

one of R₁ and R₂ is diethylamino and the other one is a hydrogen atom; or

one of R₁ and R₂ is amino and the other one is a hydrogen atom.

Other examples of compounds according to the invention are those in which P is a hydrogen, and Y is a C₂₀ alkenyl with 5 methylene interrupted double bonds located in Z configuration, wherein:

one of R₁ and R₂ is methyl and the other one is a hydrogen atom;

one of R₁ and R₂ is ethyl and the other one is a hydrogen atom;

one of R₁ and R₂ is propyl and the other one is a hydrogen atom;

one of R₁ and R₂ is methoxy and the other one is a hydrogen atom;

one of R₁ and R₂ is ethoxy and the other one is a hydrogen atom;

one of R₁ and R₂ is propoxy and the other one is a hydrogen atom;

one of R₁ and R₂ is benzyl and the other one is a hydrogen atom;

one of R₁ and R₂ is thiomethyl and the other one is a hydrogen atom;

one of R₁ and R₂ is thioethyl and the other one is a hydrogen atom; or

one of R₁ and R₂ is thiopropyl and the other one is a hydrogen atom.

Further examples of compounds according to the invention are those in which P is a hydrogen, and Y is a C₁₈ alkenyl with 5 methylene interrupted double bonds, located in Z configuration, wherein:

one of R₁ and R₂ is methyl and the other one is a hydrogen atom;

one of R₁ and R₂ is ethyl and the other one is a hydrogen atom;

one of R₁ and R₂ is propyl and the other one is a hydrogen atom;

one of R₁ and R₂ is methoxy and the other one is a hydrogen atom;

one of R₁ and R₂ is ethoxy and the other one is a hydrogen atom;

one of R₁ and R₂ is propoxy and the other one is a hydrogen atom;

one of R₁ and R₂ is thiomethyl and the other one is a hydrogen atom;

one of R₁ and R₂ is thioethyl and the other one is a hydrogen atom;

one of R₁ and R₂ is thiopropyl and the other one is a hydrogen atom;

one of R₁ and R₂ is ethylamino and the other one is a hydrogen atom;

one of R₁ and R₂ is benzyl and the other one is a hydrogen atom;

one of R₁ and R₂ is diethylamino and the other one is a hydrogen atom; or

one of R₁ and R₂ is amino and the other one is a hydrogen atom.

Additional examples of compounds according to the invention are those in which P is a hydrogen, and Y is a C₁₆ alkenyl with 3 methylene interrupted double bonds, located in Z configuration, wherein:

one of R₁ and R₂ is methyl and the other one is a hydrogen atom;

one of R₁ and R₂ is ethyl and the other one is a hydrogen atom;

one of R₁ and R₂ is propyl and the other one is a hydrogen atom;

one of R₁ and R₂ is methoxy and the other one is a hydrogen atom;

one of R₁ and R₂ is ethoxy and the other one is a hydrogen atom;

one of R₁ and R₂ is propoxy and the other one is a hydrogen atom;

one of R₁ and R₂ is thiomethyl and the other one is a hydrogen atom;

one of R₁ and R₂ is thioethyl and the other one is a hydrogen atom;

one of R₁ and R₂ is thiopropyl and the other one is a hydrogen atom;

one of R₁ and R₂ is ethylamino and the other one is a hydrogen atom;

one of R₁ and R₂ is benzyl and the other one is a hydrogen atom;

one of R₁ and R₂ is diethylamino and the other one is a hydrogen atom; or

one of R₁ and R₂ is amino and the other one is a hydrogen atom.

In the omega-3 lipid compound according to formula (I) of the present invention, R₁ and R₂ may be the same or different. When they are different, the compounds of formula (I) are capable of existing in stereoisomeric forms. It will be understood that the invention encompasses all optical isomers of the compounds of formula (I) or mixtures thereof, including racemates. Therefore, the present invention includes, where R₁ is different from R₂, compounds of formula (I) that are racemic or enantiomerically pure, either as the (R) or the (S) enantiomer.

The present invention also relates to an omega-3 compound according of formula (I) for use as a medicament or for diagnostic purposes, for instance for use in positron emission tomography (PET). In addition the compounds and compositions according to the present invention can be used as cosmetic products, in particular as a topical preparation for skin. Those preparation can be used for various purposes, including the treatment of psorasis.

Further, the present invention relates to a pharmaceutical composition comprising an omega-3 lipid compound according to formula (I). The pharmaceutical composition may comprise a pharmaceutically acceptable carrier, excipient or diluent, or any combination thereof, and is suitably formulated for oral administration, e.g. in the form of a capsule or a sachet. A suitable daily dosage of the compound according to formula (I) is 5 mg to 10 g of said compound; 50 mg to 1 g of said compound, or 50 mg to 200 mg of said compound.

The present invention also relates to lipid composition comprising an omega-3 lipid compound according to formula (I). Suitably, said omega-3 lipid compound is present in a concentration of at least 60% by weight, or at least 80% by weight of the lipid composition. The lipid composition may further comprise omega-3 fatty alcohols, or pro-drugs thereof, chosen from (all-Z)-5,8,11,14,17-eicosapentaen-1-ol (EPA), (all-Z)-4,7,10,13,16,19-docosahexaen-1-ol acid (DHA), (all-Z)-6,9,12,15,18-heneicosapentaen-1-ol acid (HPA), and/or (all-Z)-7,10,13,16,19-docosapentaen-1-ol (DPA), or derivatives thereof, i.e. presented in their 2-substituted form, and/or a pharmaceutically acceptable antioxidant, e.g. tocopherol.

Further, the invention relates to the use of an omega-3 lipid compound according to formula (I) for the production of a medicament for the following:
activation or modulation of at least one of the human peroxisome proliferator-activated receptor (PPAR) isoforms, wherein said peroxisome proliferator-activated receptor (PPAR) is peroxisome proliferator-activated receptor (PPAR)α and/or γ.
the treatment and/or the prevention of peripheral insulin resistance and/or a diabetic condition.
reduction of plasma insulin, blood glucose and/or serum triglycerides.
the treatment and/or the prevention of type 2 diabetes.
the prevention and/or treatment of elevated triglyceride levels, LDL cholesterol levels, and/or VLDL cholesterol levels.
the prevention and/or treatment of a hyperlipidemic condition, e.g. hypertriglyceridemia (HTG).
increasing serum HDL levels in humans.
the treatment and/or the prevention of obesity or an overweight condition.
reduction of body weight and/or for preventing body weight gain.
the treatment and/or the prevention of a fatty liver disease, e.g. non-alcoholic fatty liver disease (NAFLD).
treatment of insulin resistance, hyperlipidemia and/or obesity or an overweight condition.
the production of a medicament for the treatment and/or the prevention of an inflammatory disease or condition.

The invention also relates to methods for the treatment and/or prevention of the conditions listed above, comprising administering to a mammal in need thereof a pharmaceutically active amount of a compound according to formula (I).

In addition, the present invention encompasses methods for manufacturing omega-3 lipid compounds according to formula (I).

### DETAILED DESCRIPTION OF THE INVENTION

Research shows that introduction of a substituent in the α-position of polyunsaturated fatty acids increases their affinities to nuclear receptors and, in particular, to the PPARs. Because the PPARs are key regulators of energy homeostasis and inflammation, much research has been directed towards development of synthetic PPAR ligands.

The carboxylic acid functional group of the PUFAs is important to target binding in the PPARs, but this ionizable group may hinder the drug from crossing the cell membranes of the gut wall. Accordingly, carboxylic acids functional groups in drugs are often protected as an ester. The less polar ester group can cross the fatty cell membranes, and once in the bloodstream it can be hydrolyzed back to the free acid by esterases in the blood.

It is also a possible that plasma enzymes do not hydrolyze these esters fast enough and that the conversion of ester to free acid predominantly takes place downstream in liver. The same occurs for ethyl esters of polyunsaturated fatty acids that are hydrolyzed to a free acid in vivo.

Because 2-substituted polyunsaturated fatty acid derivatives have the potential of being used for therapeutic purposes, compounds according to the present invention are novel pro-drugs of α-substituted fatty acids. These pro-drugs may have improved therapeutically activity, increased bioavailability and ability to cross the cell membrane.

Each PPAR receptor subtype exhibits a distinct pattern of expression and overlapping but distinct biological activities. Whereas PPAR-α and PPAR-γ are predominantly present in the liver and adipose tissue, respectively, PPAR-δ is ubiquitously expressed. Because of the different distribution of PPAR receptor subtypes, drugs targeting these receptors should target the tissue where the desired receptor is expressed. Variation of the functional group in addition to variation in chain length and number of double bounds might give a kind of tissue specificity to the compounds of the present invention.

Exemplary embodiments include omega-3 polyunsaturated alcohols, or pro-drugs thereof, which are substituted in the 2 position. Moreover, a lipid composition, comprising omega-3 compounds according to the invention, may reduce triglyceride levels, and cholesterol and at the same time increase HDL levels. The pharmaceutically product according to the invention may also give an increased effect on inflammatory diseases, neural development and visual functions.

Nomenclature and Terminology

Fatty acids are straight chain hydrocarbons possessing a carboxyl (COOH) group at one end (α) and (usually) a methyl group at the other (ω) end. Fatty acids are named by the position of the first double bond from the ω end. The term ω-3 (omega-3) signifies that the first double bond exists as the third carbon-carbon bond from the terminal CH₃ end (ω) of the carbon chain. However, according to chemical nomenclature convention, the numbering of the carbon atoms starts from the α end.

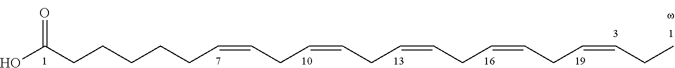

According to the present invention, the carboxylic group has been replaced by a new functional group in the form of an alcohol, or a pro-drug thereof.

As used herein, the expression “methylene interrupted double bonds” relates to the case when a methylene group is located between two separate double bonds in a carbon chain of an omega-3 lipid compound.

Throughout this specification, the terms “2-substituted”, substituted in position 2, and “substituted at carbon 2, counted from the functional group of the omega-3 lipid compound” refers to a substitution at the carbon atom denoted 2 in accordance with the above numbering of the carbon chain. Alternatively, such a substitution may be called an “2-substitution”.

Throughout this specification, the term “omega-3 lipid compound” (corresponding to ω-3, or n-3) relates to a lipid compound having the first double bond at the third carbon-carbon bond from the ω end of the carbon chain, as defined above.

The basic idea of the present invention is an omega-3 lipid compound of formula (I):

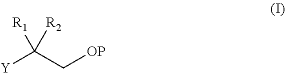

wherein

R₁ and R₂ are the same or different and are chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

P represents a hydrogen atom, or

P represents;

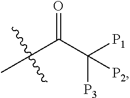

wherein P₁, P₂, and P₃ are chosen from a hydrogen atom, an alkyl group, and a C₁₄-C₂₂ alkenyl group, wherein the alkyl and alkenyl groups are optionally substituted with a hydroxy group, or

P represents;

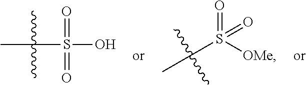

P represents;

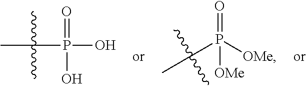

P represents;

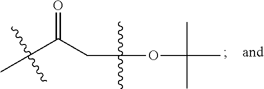

Y is a C₁₄-C₂₂ alkenyl group with at least one double bond, having E and/or Z configuration;

or any pharmaceutically acceptable complex, solvate, salt or pro-drug thereof, with the proviso that R₁ and R₂ are not simultaneously a hydrogen atom.

The resulting compound is an 2-substituted omega-3 lipid compound, i.e. an omega-3 lipid compound substituted in position 2 of the carbon atom, counted from the carbonyl end. More particularly, the resulting compound is an 2-substituted polyunsaturated omega-3 alcohol, or a pro-drug thereof. Exemplary, pro-drugs relates to omega-3 lipid compounds of formula (II):

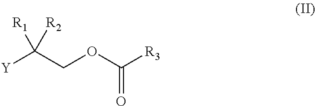

wherein:

R₃ is a C₁-C₆ alkyl.

Other exemplary pro-drugs include:

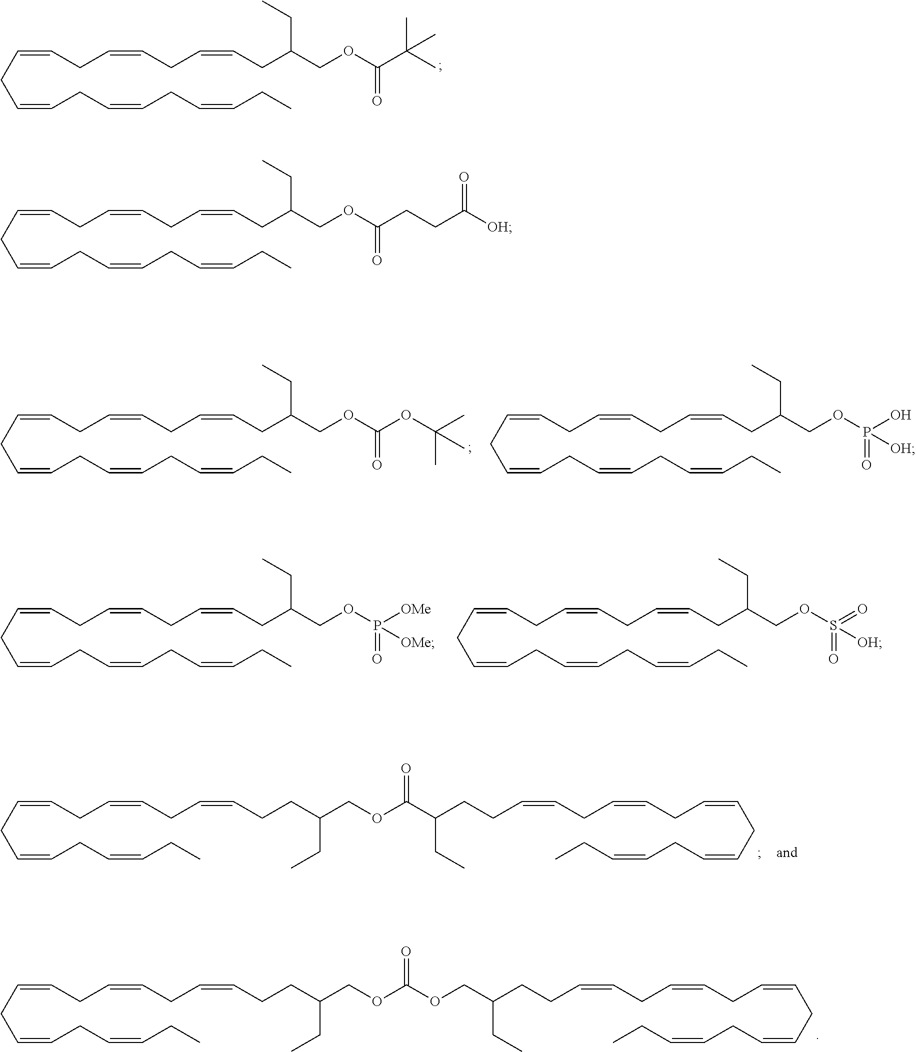

Other exemplary embodiments include the following omega-3 derivatives substituted in position 2:
(all-Z)-4,7,10,13,16,19-docosahexaen-1-ol,
(all-Z)-5,8,11,14,17-eicosapentaen-1-ol,
(all-Z)-9,12,15-octadecatrien-1-ol,
(all-Z)-6,9,12,15-octadecatetraen-1-ol,
(all-Z)-7,10,13,16,19-docosapentaen-1-ol,
(all-Z)-11,14,17-eicosatrien-1-ol,
(all-Z)-6,9,12,15,18,21-tetracosahexaen-1-ol,
(4E,8Z,11Z,14Z,17Z)-eicosapentaen-1-ol,
(5E,8Z,11Z,14Z,17Z)-eicosapentaen-1-ol,
(all-Z)-8,11,14,17-eicosatetraen-1-ol, and
(4E,7Z,10Z,13Z,16Z,19Z)-docosahexaen-1-ol.

Among the possible substituents listed above for R₁ and R₇, lower alkyl groups, in particular methyl and ethyl groups, are preferred embodiments. Other exemplary substitutents such as lower alkoxy or lower alkylthio groups, e.g. having 1-3 carbon atoms. The substitution of either R₁ or R₂ with any one of these substituents, while the other one is hydrogen, is believed to provide the most efficient result.

Exemplary omega-3 polyunsaturated lipids which can be substituted in the position include (all-Z)-4,7,10,13,16,19-docosahexaen-1-ol, (all-Z)-5,8,11,14,17-eicosapentaen-1-ol, (all-Z)-7,10,13,16,19-docosapentaen-1-ol and (all-Z)-9,12,15-octadecatrien-1-ol. Suitable substituents include a hydrogen atom and lower alkyl groups, preferably having 1-3 carbon atoms, and more preferably 2-3 carbon atoms.

Omega-3 lipid compounds, i.e. substituted omega-3 alcohols and potential pro-drugs thereof, according to the invention are divided into the following categories A-H;

Category A

(all-Z)-4,7,10,13,16,19-docosahexaen-1-ol, or a pro-drug thereof, substituted in position 2 counted from the functional group:

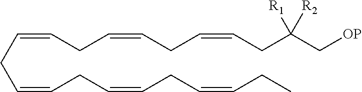

Y═ is C₂₀ alkenyl with 6 methylene interrupted double bonds in Z-configuration.

Category B

(all-Z)-5,8,11,14,17-eicosapentaen-1-ol, or a pro-drug thereof, substituted in position 2 counted from the functional group:

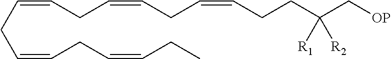

Y═C₁₈ alkenyl with 5 methylene interrupted double bonds in Z-configuration

Category C

(all-Z)-9,12,15-octadecatrien-1-ol, or a pro-drug thereof, substituted in position 2 counted from the functional group:

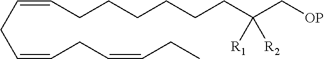

Y═C₁₆ alkenyl with 3 methylene interrupted double bonds in Z-configuration.

Category D

(all-Z)-7,10,13,16,19-docosapentaen-1-ol, a pro-drug, thereof substituted in position 2 counted from the functional group:

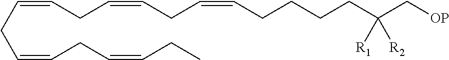

Y═C₂₀ alkenyl with 5 methylene interrupted double bonds in Z-configuration.

Category E

(4E,8Z,11Z,14Z,17Z)-eicosapentaen-1-ol, or a pro-drug thereof, substituted in position 2 counted from the functional group:

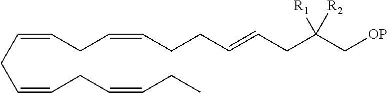

Y═C₁₈alkenyl with 5 double bonds

Category F

(all-Z)-11,14,17-eicosatrien-1-ol, or a pro-drug thereof, substituted in position 2 counted from the functional group:

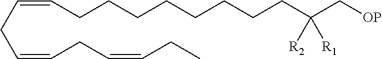

Y═C₁₈ alkenyl with 3 methylene interrupted double bonds in Z-configuration

Category G

(4E,7Z,10Z,13Z,16Z,19Z)-docosahexaen-1-ol, or pro-drugs thereof, substituted in position 2 counted from the functional group:

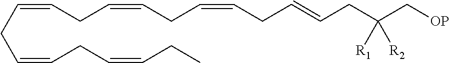

Y═C₂₀ alkenyl with 6 double bonds.

Category H

(5E,8Z,11Z,14Z,17Z)-eicosapentaen-1-ol, or pro-drugs thereof, substituted in position 2 counted from the functional group:

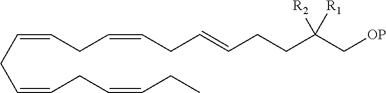

Y═C₁₈ alkenyl with 5 double bonds,
wherein P is —CH₂COOH.

Category I

α-Substituted omega-3 lipid compounds, wherein P represents:

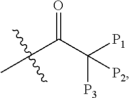

wherein P₁, P₂, and P₃ are each a hydrogen atom, and wherein R₁, R₂, and Y are hereinabove defined.

Category J

α-Substituted omega-3 lipid compounds, wherein P represents:

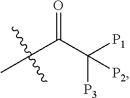

wherein P₁, P₂, and P₃ are each a methyl group, and wherein R₁, R₂, and Y are hereinabove defined.

Category K

α-Substituted omega-3 lipid compounds, wherein P represents:

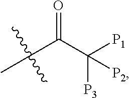

wherein P₁ is an ethyl group substituted with a hydroxy group, P₂, and P₃ are each a hydrogen atom, and wherein R₁, R₂, and Y are hereinabove defined.

Category L

α-Substituted omega-3 lipid compounds, wherein P represents:

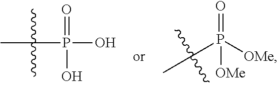

wherein R₁, R₂, and Y are hereinabove defined.

Category M

α-Substituted omega-3 lipid compounds, wherein P represents:

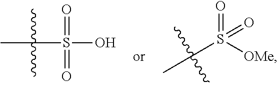

wherein R₁, R₂, and Y are hereinabove defined.

Category N

α-Substituted omega-3 lipid compounds, wherein P represents:

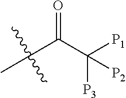

wherein P₁, P₂, and P₃ are each a methyl group.

Category O

α-Substituted omega-3 lipid compounds, wherein P represents:

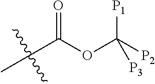

wherein P₁ is an alkenyl group, and P₂, and P₃ are each a hydrogen atom, and wherein R₁, R₂, and Y are hereinabove defined.

### Category A

### Examples (1)-(8)

For all examples (1)-(8):
P is a hydrogen.
Y is a C₂₀ alkenyl with 6 methylene interrupted double bonds.

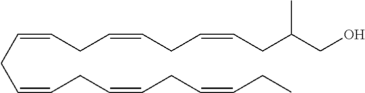

### (all-Z)-2-methyl-4,7,10,13,16,19-docosahexaen-1-ol (1)

R₁=methyl, and R₂=a hydrogen atom, or
R₂=methyl, and R₁=a hydrogen atom

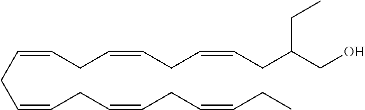

### (all-Z)-2-ethyl-4,7,10,13,16,19-docosahexaen-1-ol (2)

R₁=ethyl, and R₂=a hydrogen atom, or
R₂=ethyl, and R₁=a hydrogen atom

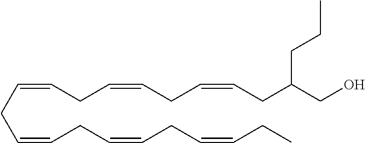

### (all-Z)-2-propyl-4,7,10,13,16,19-docosahexaen-1-ol (3)

R₁=propyl, and R₂=a hydrogen atom, or
R₂=propyl, and R₁=a hydrogen atom

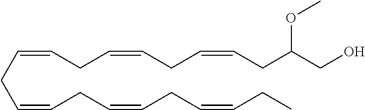

### (all-Z)-2-methoxy-4,7,10,13,16,19-docosahexaen-1-ol (4)

R₁=methoxy, and R₂=a hydrogen atom, or
R₂=methoxy, and R₁=a hydrogen atom

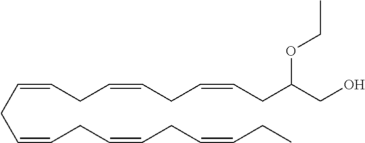

### (all-Z)-2-ethoxy-4,7,10,13,16,19-docosahexaen-1-ol (5)

R₁=ethoxy, and R₂=a hydrogen atom, or
R₂=ethoxy, and R₁=a hydrogen atom

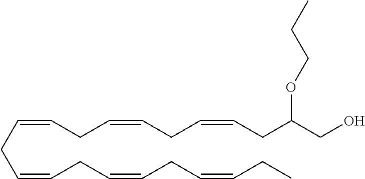

### (all-Z)-2-propoxy-4,7,10,13,16,19-docosahexaen-1-ol (6)

R₁=propoxy, and R₂=a hydrogen atom, or
R₂=propoxy, and R₁=a hydrogen atom

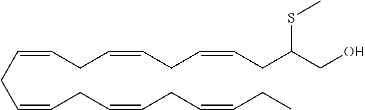

### (all-Z)-2-thiomethyl-4,7,10,13,16,19-docosahexaen-1-ol (7)

R₁=thiomethyl, and R₂=a hydrogen atom, or
R₂=thiomethyl, and R₁=a hydrogen atom

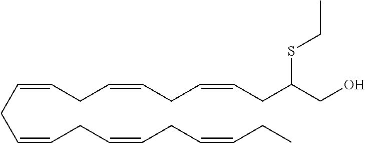

### (all-Z)-2-thioethyl-4,7,10,13,16,19-docosahexaen-1-ol (8)

R₁=thioethyl, and R₂=a hydrogen atom, or
R₂=thioethyl, and R₁=a hydrogen atom

### Category B

### Examples (9)-(17)

For all examples (9)-(17):
P=hydrogen atom
Y═C₁₈ alkenyl with 5 methylene interrupted double bonds in Z-configuration

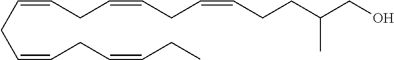

### (all-Z)-2-methyl-5,8,11,14,17-eicosapentaen-1-ol (9)

R₁=methyl, and R₂=a hydrogen atom, or
R₂=methyl, and R₁=a hydrogen atom

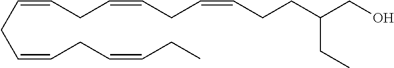

### (all-Z)-2-ethyl-5,8,11,14,17-eicosapentaen-1-ol (10)

R₁=ethyl, and R₂=a hydrogen atom, or
R₂=ethyl, and R₁=a hydrogen atom

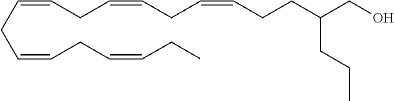

### (all-Z)-2-propyl-5,8,11,14,17-eicosapentaen-1-ol (11)

R₁=propyl, and R₂=a hydrogen atom, or
R₂=propyl, and R₁=a hydrogen atom

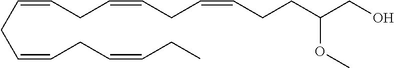

### (all-Z)-2-methoxy-5,8,11,14,17-eicosapentaen-1-ol (12)

R₁=methoxy, and R₂=a hydrogen atom, or
R₂=methoxy, and R₁=a hydrogen atom

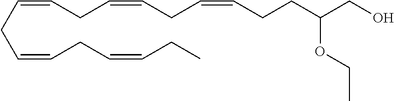

### (all-Z)-2-ethoxy-5,8,11,14,17-eicosapentaen-1-ol (13)

R₁=ethoxy, and R₂=a hydrogen atom, or
R₂=ethoxy, and R₁=a hydrogen atom

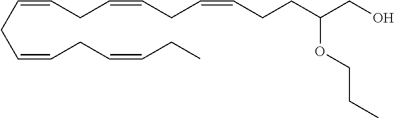

### (all-Z)-2-propoxy-5,8,11,14,17-eicosapentaen-1-ol (14)

R₁=propoxy, and R₂=a hydrogen atom, or
R₂=propoxy, and R₁=a hydrogen atom

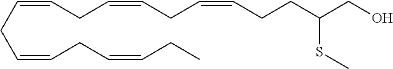

### (all-Z)-2-thiomethyl-5,8,11,14,17-eicosapentaen-1-ol (15)

R₁=methylthio, and R₂=a hydrogen atom, or
R₂=methylthio, and R₁=a hydrogen atom

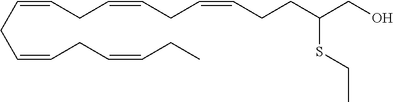

### (all-Z)-2-thioethyl-5,8,11,14,17-eicosapentaen-1-ol (16)

R₁=ethylthio, and R₂=a hydrogen atom, or
R₂=ethylthio, and R₁=a hydrogen atom

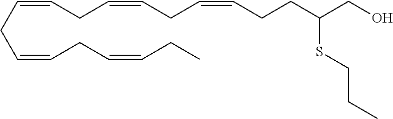

### (all-Z)-2-thiopropyl-5,8,11,14,17-eicosapentaen-1-ol (17)

R₁=propylthio, and R₂=a hydrogen atom, or
R₂=propylthio, and R₁=a hydrogen atom

### Category C

### Examples (18)-(26)

For all examples (18)-(26):
P=Hydrogen atom
Y═C₁₆ alkenyl with 3 methylene interrupted double bonds in Z-configuration

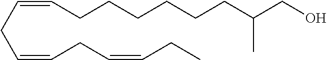

### (all-Z)-2-methyl-9,12,15-octadecatrien-1-ol (18)

R₁=methyl, and R₂=a hydrogen atom, or
R₂=methyl, and R₁=a hydrogen atom

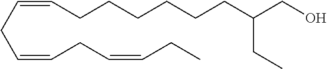

### (all-Z)-2-ethyl-9,12,15-octadecatrien-1-ol (19)

R₁=ethyl, and R₂=a hydrogen atom, or
R₂=ethyl, and R₁=a hydrogen atom

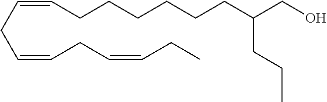

### (all-Z)-2-propyl-9,12,15-octadecatrien-1-ol (20)

R₁=propyl, and R₂=a hydrogen atom, or
R₂=propyl, and R₁=a hydrogen atom

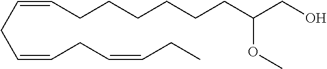

### (all-Z)-2-methoxy-9,12,15-octadecatrien-1-ol (21)

R₁=methoxy, and R₂=a hydrogen atom, or
R₂=methoxy, and R₁=a hydrogen atom

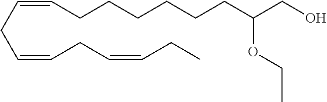

### (all-Z)-2-ethoxy-9,12,15-octadecatrien-1-o (22)

R₁=ethoxy, and R₂=a hydrogen atom, or
R₂=ethoxy, and R₁=a hydrogen atom

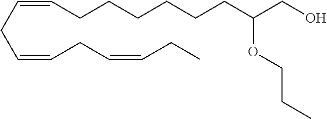

### (all-Z)-2-propoxy-9,12,15-octadecatrien-1-ol (23)

R₁=propoxy, and R₂=a hydrogen atom, or
R₂=propoxy, and R₁=a hydrogen atom

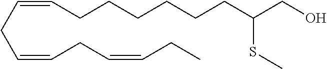

### (all-Z)-2-thiomethyl-9,12,15-octadecatrien-1-ol (24)

R₁=methylthio, and R₂=a hydrogen atom, or
R₂=methylthio, and R₁=a hydrogen atom

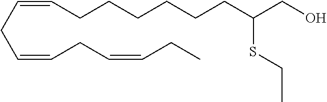

### (all-Z)-2-thioethyl-9,12,15-octadecatrien-1-ol (25)

R₁=ethylthio, and R₂=a hydrogen atom, or
R₂=ethylthio, and R₁=a hydrogen atom

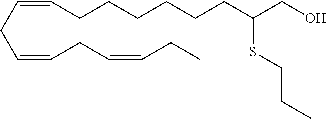

### (all-Z)-2-thiopropyl-9,12,15-octadecatrien-1-ol (26)

R₁=propylthio, and R₂=a hydrogen atom, or
R₂=propylthio, and R₁=a hydrogen atom

### Category D

### Examples (27)-(35)

For all examples (27)-(35):
P=hydrogen atom
Y═C₂₀ alkenyl with 5 methylene interrupted double bonds in Z-configuration

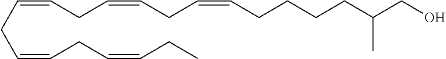

### (all-Z)-2-methyl-7,10,13,16,19-docosapentaen-1-ol (27)

R₁=methyl, and R₂=a hydrogen atom, or
R₂=methyl, and R₁=a hydrogen atom

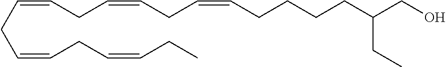

### (all-Z)-2-ethyl-7,10,13,16,19-docosapentaen-1-ol (28)

R₁=ethyl, and R₂=a hydrogen atom, or
R₂=ethyl, and R₁=a hydrogen atom

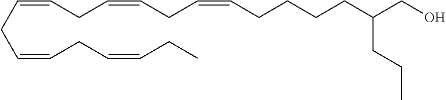

### (all-Z)-2-propyl-7,10,13,16,19-docosapentaen-1-ol (29)

R₁=propyl, and R₂=a hydrogen atom, or
R₂=propyl, and R₁=a hydrogen atom

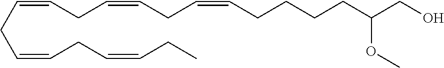

### (all-Z)-2-methoxy-7,10,13,16,19-docosapentaen-1-ol (30)

R₁=methoxy, and R₂=a hydrogen atom, or
R₂=methoxy, and R₁=a hydrogen atom

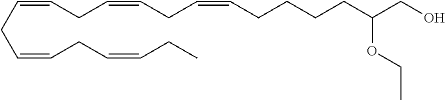

### (all-Z)-2-ethoxy-7,10,13,16,19-docosapentaen-1-ol (31)

R₁=ethoxy, and R₂=a hydrogen atom, or
R₂=ethoxy, and R₁=a hydrogen atom

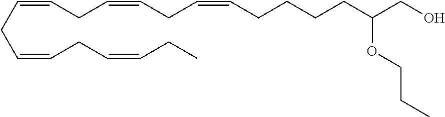

### (all-Z)-2-propoxy-7,10,13,16,19-docosapentaen-1-ol (32)

R₁=propoxy, and R₂=a hydrogen atom, or
R₂=propoxy, and R₁=a hydrogen atom

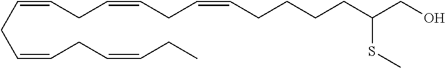

### (all-Z)-2-thiomethyl-7,10,13,16,19-docosapentaen-1-ol (33)

R₁=methylthio, and R₂=a hydrogen atom, or
R₂=methylthio, and R₁=a hydrogen atom

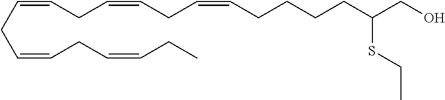

### (all-Z)-2-thioethyl-7,10,13,16,19-docosapentaen-1-ol (34)

R₁=ethylthio, and R₂=a hydrogen atom, or
R₂=ethylthio, and R₁=a hydrogen atom

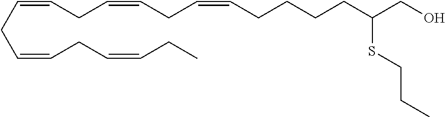

### (all-Z)-2-thiopropyl-7,10,13,16,19-docosapentaen-1-ol (35)

R₁=propylthio, and R₂=a hydrogen atom, or
R₂=propylthio, and R₁=a hydrogen atom

### Category E

### Examples (36)-(44)

For all examples (36)-(44)
Y═C₁₈ alkenyl with 5 double bonds.
P=hydrogen atom

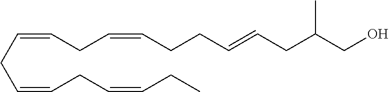

### (4E,8Z,11Z,14Z,17Z)-2-methyl-eicosapentaen-1-ol (36)

R₁=methyl, and R₂=a hydrogen atom, or
R₂=methyl, and R₁=a hydrogen atom

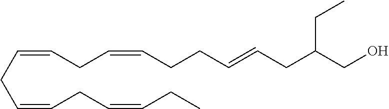

### (4E,8Z,11Z,14Z,17Z)-2-ethyl-eicosapentaen-1-ol (37)

R₁=ethyl, and R₂=a hydrogen atom, or
R₂=ethyl, and R₁=a hydrogen atom

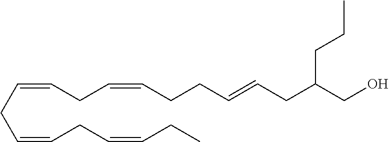

### (4E,8Z,11Z,14Z,17Z)-2-propyl-eicosapentaen-1-ol (38)

R₁=propyl, and R₂=a hydrogen atom, or
R₁=propyl, and R₁=a hydrogen atom

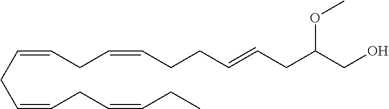

### (4E,8Z,11Z,14Z,17Z)-2-methoxy-eicosapentaen-1-ol (39)

R₁=methoxy, and R₂=a hydrogen atom, or
R₂=methoxy, and R₁=a hydrogen atom

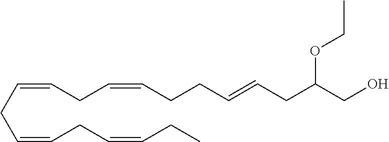

### (4E,8Z,11Z,14Z,17Z)-2-ethoxy-eicosapentaen-1-ol (40)

R₁=ethoxy, and R₂=a hydrogen atom, or
R₂=ethoxy, and R₁=a hydrogen atom

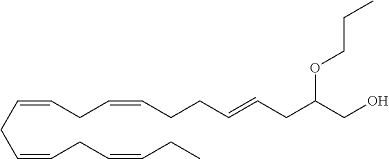

### (4E,8Z,11Z,14Z,17Z)-2-propoxy-eicosapentaen-1-ol (41)

R₁=propoxy, and R₂=a hydrogen atom, or
R₂=propoxy, and R₁=a hydrogen atom

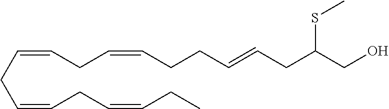

### (4E,8Z,11Z,14Z,17Z)-2-thiomethyl-eicosapentaen-1-ol (42)

R₁=methylthio, and R₂=a hydrogen atom, or
R₂=methylthio, and R₁=a hydrogen atom

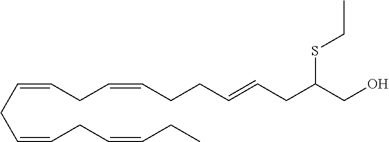

### (4E,8Z,11Z,14Z,17Z)-2-thioethyl-eicosapentaen-1-ol (43)

R₁=ethylthio, and R₂=a hydrogen atom, or
R₂=ethylthio, and R₁=a hydrogen atom

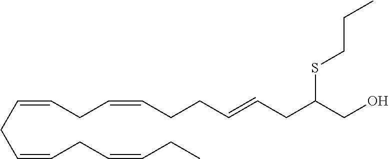

### (4E,8Z,11Z,14Z,17Z)-2-thiopropyl-eicosapentaen-1-ol (44)

R₁=propylthio, and R₂=a hydrogen atom, or
R₂=propylthio, and R₁=a hydrogen atom

### Category F

### Examples (45)-(54)

For all examples
Y═C₁₈ alkenyl with 3 methylene interrupted double bonds in Z-configuration
P=hydrogen atom

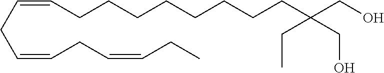

### (all-Z)-2-ethyl,2-hydroxymethyl-11,14,17-eicosatrien-1-ol (45)

R₁=ethyl, and R₂=hydroxy, or
R₂=hydroxy, and R₁=ethyl

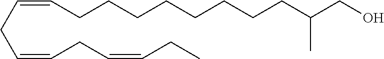

### (all-Z)-2-methyl-11,14,17-eicosatrien-1-ol (46)

R₁=methyl, and R₂=a hydrogen atom, or
R₂=methyl, and Rₜ=a hydrogen atom

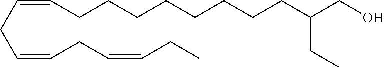

### (all-Z)-2-ethyl-11,14,17-eicosatrien-1-ol (47)

R₁=ethyl, and R₂=a hydrogen atom, or
R₂=ethyl, and R₁=a hydrogen atom

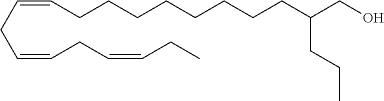

### (all-Z)-2-propyl-11,14,17-eicosatrien-1-ol (48)

R₁=propyl, and R₂=a hydrogen atom, or
R₂=propyl, and R₁=a hydrogen atom

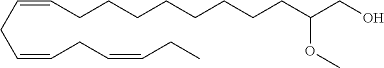

### (all-Z)-2-methoxy-11,14,17-eicosatrien-1-ol (49)

R₁=methoxy, and R₂=a hydrogen atom, or
R₂=methoxy, and R₁=a hydrogen atom

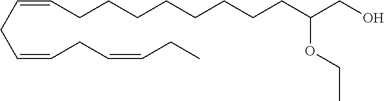

### (all-Z)-2-ethoxy-11,14,17-eicosatrien-1-ol (50)

R₁=ethoxy, and R₂=a hydrogen atom, or
R₂=ethoxy, and R₁=a hydrogen atom

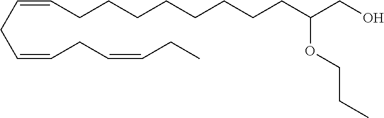

### (all-Z)-2-propoxy-11,14,17-eicosatrien-1-ol (51)

R₁=propoxy, and R₂=a hydrogen atom, or
R₂=propoxy, and R₁=a hydrogen atom

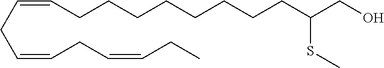

### (all-Z)-2-thiomethyl-11,14,17-eicosatrien-1-ol (52)

R₁=methylthio, and R₂=a hydrogen atom, or
R₂=methylthio, and R₁=a hydrogen atom

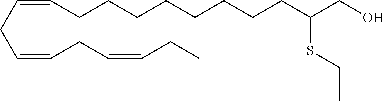

### (all-Z)-2-thioethyl-11,14,17-eicosatrien-1-ol (53)

R₁=ethylthio, and R₂=a hydrogen atom, or
R₂=ethylthio, and R₁=a hydrogen atom

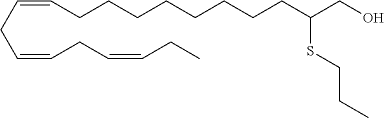

### (all-Z)-2-thiopropyl-11,14,17-eicosatrien-1-ol (54)

R₁=propylthio, and R₂=a hydrogen atom, or
R₂=propylthio, and R₁=a hydrogen atom

### Category G

### Examples (55)-(63)

For all examples (55)-(63):
Y═C₂₀ alkene with 6 double bonds
P=hydrogen atom

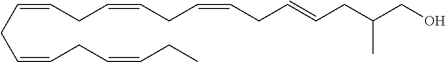

### 4E,7Z,10Z,13Z,16Z,19Z-2-methyl-docosahexaen-1-ol (55)

R₁=methyl, and R₂=a hydrogen atom, or
R₂=methyl, and R₁=a hydrogen atom

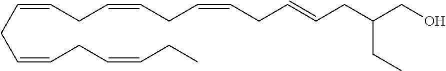

### (4E,7Z,10Z,13Z,16Z,19Z)-2-ethyl-docosahexaen-1-ol (56)

R₁=ethyl, and R₂=a hydrogen atom, or
R₂=ethyl, and R₁=a hydrogen atom

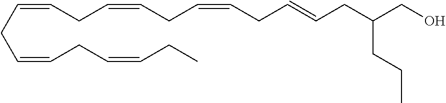

### (4E,7Z,10Z,13Z,16Z,19Z)-2-propyl-docosahexaen-1-ol (57)

R₁=propyl, and R₂=a hydrogen atom, or
R₂=propyl, and R₁=a hydrogen atom

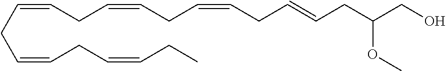

### (4E,7Z,10Z,13Z,16Z,19Z)-2-methoxy-docosahexaen-1-ol (58)

R₁=methoxy, and R₂=a hydrogen atom, or
R₂=methoxy, and R₁=a hydrogen atom

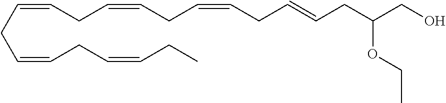

### (4E,7Z,10Z,13Z,16Z,19Z)-2-ethoxy-1-docosahexaen-1-ol (59)

R₁=ethoxy, and R₂=a hydrogen atom, or
R₂=ethoxy, and R₁=a hydrogen atom

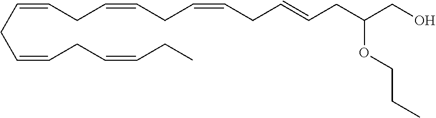

### (4E,7Z,10Z,13Z,16Z,19Z)-2-propoxy-docosahexaen-1-ol (60)

R₁=propoxy, and R₂=a hydrogen atom, or
R₂=propoxy, and R₁=a hydrogen atom

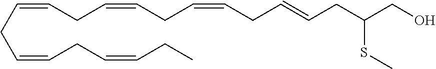

### (4E,7Z,10Z,13Z,16Z,19Z)-2-thiomethyl-docosahexaen-1-ol (61)

R₁=methylthio, and R₂=a hydrogen atom, or
R₂=methylthio, and R₁=a hydrogen atom

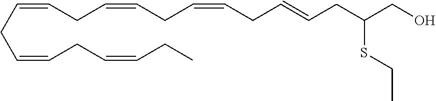

### (4E,7Z,10Z,13Z,16Z,19Z)-2-thioethyl-docosahexaen-1-ol (62)

R₁=ethylthio, and R₂=a hydrogen atom, or
R₂=ethylthio, and R₁=a hydrogen atom

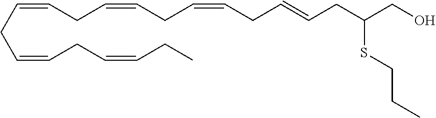

### (4E,7Z,10Z,13Z,16Z,19Z)-2-thiopropyl-docosahexaen-1-ol (63)

R₁=propylthio, and R₂=a hydrogen atom, or
R₂=propylthio, and R₁=a hydrogen atom

### Category H

### Examples (64)-(66)

For all examples (64)-(66):
Y═C₁₈ alkene with 5 double bonds
P=hydrogen atom

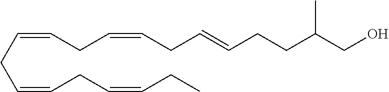

### 5E,8Z,11Z,14Z,17Z-2-methyl-eicosapentaen-1-ol (64)

R₁=methyl, and R₂=a hydrogen atom, or
R₂=methyl, and R₁=a hydrogen atom

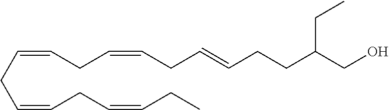

### 5E,8Z,11Z,14Z,17Z-2-ethyl-eicosapentaen-1-ol (65)

R₁=ethyl, and R₂=a hydrogen atom, or
R₂=ethyl, and R₁=a hydrogen atom

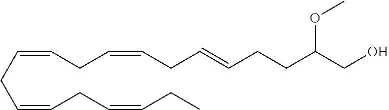

### 5E,8Z,11Z,14Z,17Z-2-ethoxy-eicosapentaen-1-ol (66)

R₁=ethoxy and R₂=a hydrogen atom, or
R₂=ethoxy, and R₁=a hydrogen atom

### Category I

### Examples (67)-(69)

For all examples (67)-(69):

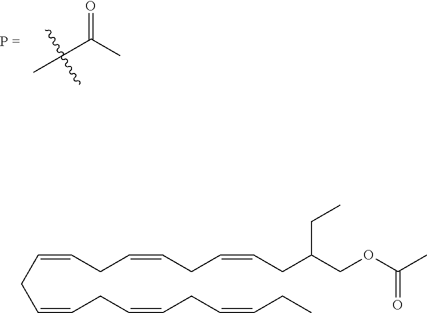

### (all-Z)-2-ethyl-4,7,10,13,16,19-docosahexaen-1-ol acetate (67)

Y is a C₂₀ alkenyl with 6 methylene interrupted double bonds
R₁=ethyl, and R₂=a hydrogen atom, or
R₂=ethyl, and R₁=a hydrogen atom

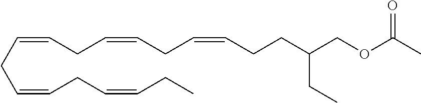

### (all-Z)-2-ethyl-5,8,11,14,17-eicosapentaen-1-ol acetate (68)

Y═C₁₈ alkenyl with 5 methylene interrupted double bonds in Z-configuration
R₁=ethyl, and R₂=a hydrogen atom, or
R₂=ethyl, and R₁=a hydrogen atom

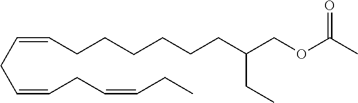

### (all-Z)-2-ethyl-9,12,15-octadecatrien-1-ol acetate (69)

Y═C₁₆ alkenyl with 3 methylene interrupted double bonds in Z-configuration
R₁=ethyl, and R₂=a hydrogen atom, or
R₂=ethyl, and R₁=a hydrogen atom

### Category J

### Examples (70)-(72)

For all examples (70)-(72):

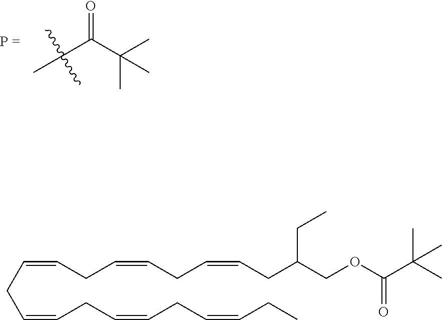

### (all-Z)-2-ethyl-4,7,10,13,16,19-docosahexaen-1-ol pivaloate (70)

Y is a C₂₀ alkenyl with 6 methylene interrupted double bonds
R₁=ethyl, and R₂=a hydrogen atom, or
R₂=ethyl, and R₁=a hydrogen atom

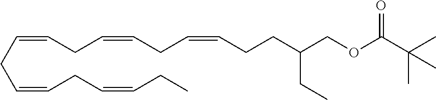

### (all-Z)-2-ethyl-5,8,11,14,17-eicosapentaen-1-ol pivaloate (71)

Y═C₁₈ alkenyl with 5 methylene interrupted double bonds in Z-configuration
R₁=ethyl, and R₂=a hydrogen atom, or
R₂=ethyl, and R₁=a hydrogen atom

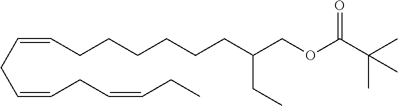

### (all-Z)-2-ethyl-9,12,15-octadecatrien-1-ol pivaloate (72)

Y═C₁₆ alkenyl with 3 methylene interrupted double bonds in Z-configuration
R₁=ethyl, and R₂=a hydrogen atom, or
R₂=ethyl, and R₁=a hydrogen atom

### Category K

### Examples (73)-(75)

For all examples (73)-(75):

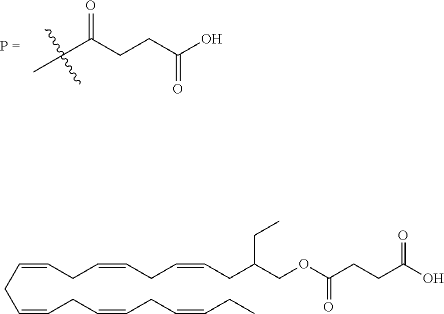

### (all-Z)-2-ethyl-4,7,10,13,16,19-docosahexaen-1-ol hemisuccinate (73)

Y is a C₂₀ alkenyl with 6 methylene interrupted double bonds
R₁=ethyl, and R₂=a hydrogen atom, or
R₂=ethyl, and R₁=a hydrogen atom

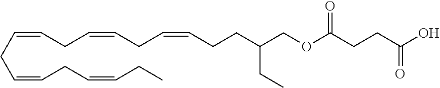

### (all-Z)-2-ethyl-5,8,11,14,17-eicosapentaen-1-ol hemisuccinate (74)

Y═C₁₈ alkenyl with 5 methylene interrupted double bonds in Z-configuration
R₁=ethyl, and R₂=a hydrogen atom, or
R₂=ethyl, and R₁=a hydrogen atom

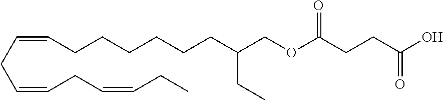

### (all-Z)-2-ethyl-9,12,15-octadecatrien-1-ol hemisuccinate (75)

Y═C₁₆ alkenyl with 3 methylene interrupted double bonds in Z-configuration
R₁=ethyl, and R₂=a hydrogen atom, or
R₂=ethyl, and R₁=a hydrogen atom

### Category L

### Examples (76)-(78)

For all examples (76)-(78):

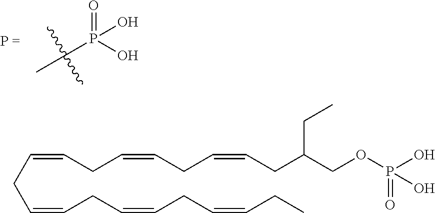

### (all-Z)-2-ethyl-4,7,10,13,16,19-docosahexaen-1-ol phosphonate (76)

Y is a C₂₀ alkenyl with 6 methylene interrupted double bonds
R₁=ethyl, and R₂=a hydrogen atom, or
R₂=ethyl, and R₁=a hydrogen atom

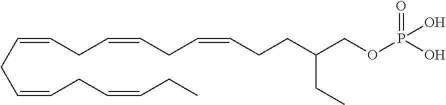

### (all-Z)-2-ethyl-5,8,11,14,17-eicosapentaen-1-ol phosphonate (77)

Y═C₁₈ alkenyl with 5 methylene interrupted double bonds in Z-configuration
R₁=ethyl, and R₂=a hydrogen atom, or
R₂=ethyl, and R₁=a hydrogen atom

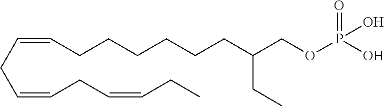

### (all-Z)-2-ethyl-9,12,15-octadecatrien-1-ol phosphonate (78)

Y═C₁₆ alkenyl with 3 methylene interrupted double bonds in Z-configuration
R₁=ethyl, and R₂=a hydrogen atom, or
R₂=ethyl, and R₁=a hydrogen atom

### Category M

### Examples (79)-(81)

For all examples (79)-(81):

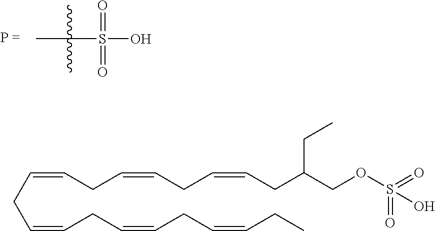

### (all-Z)-2-ethyl-4,7,10,13,16,19-docosahexaen-1-ol sulphonate (79)

Y is a C₂₀ alkenyl with 6 methylene interrupted double bonds
R₁=ethyl, and R₂=a hydrogen atom, or
R₂=ethyl, and R₁=a hydrogen atom

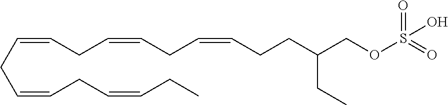

### (all-Z)-2-ethyl-5,8,11,14,17-eicosapentaen-1-ol sulphonate (80)

Y═C₁₈ alkenyl with 5 methylene interrupted double bonds in Z-configuration
R₁=ethyl, and R₂=a hydrogen atom, or
R₂=ethyl, and R₁=a hydrogen atom

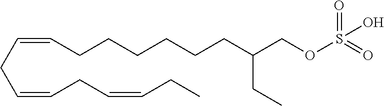

### (all-Z)-2-ethyl-9,12,15-octadecatrien-1-ol sulphonate (81)

Y═C₁₆ alkenyl with 3 methylene interrupted double bonds in Z-configuration
R₁=ethyl, and R₂=a hydrogen atom, or
R₂=ethyl, and R₁=a hydrogen atom

### Category N

### Examples (82)-(84)

For all examples (82)-(84):

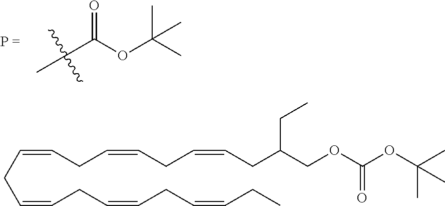

### (all-Z)-2-ethyl-4,7,10,13,16,19-docosahexaen-1-ol t-butyl carbonate (82)

Y is a C₂₀ alkenyl with 6 methylene interrupted double bonds
R₁=ethyl, and R₂=a hydrogen atom, or
R₂=ethyl, and R₁=a hydrogen atom

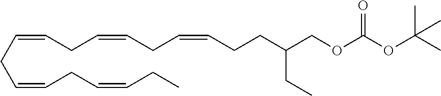

### (all-Z)-2-ethyl-5,8,11,14,17-eicosapentaen-1-ol t-butyl carbonate (83)

Y═C₁₈ alkenyl with 5 methylene interrupted double bonds in Z-configuration
R₁=ethyl, and R₂=a hydrogen atom, or
R₂=ethyl, and R₁=a hydrogen atom

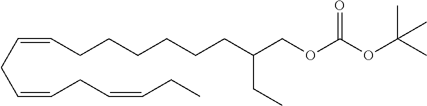

### (all-Z)-2-ethyl-9,12,15-octadecatrien-1-ol t-butyl carbonate (84)

Y═C₁₆ alkenyl with 3 methylene interrupted double bonds in Z-configuration
R₁=ethyl, and R₂=a hydrogen atom, or
R₂=ethyl, and R₁=a hydrogen atom

### Category O

### Examples (85)-(86)

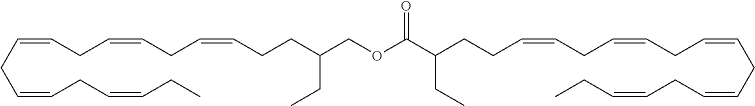

### (all-Z)-2-ethyl-5,8,11,14,17-eicosapentaen-1-ol (all-Z)-2-ethyl-5,8,11,14,17-eicosapenatenoate (85)

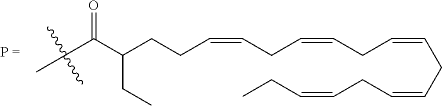

Y═C₁₈ alkenyl with 5 methylene interrupted double bonds in Z-configuration
R₁=ethyl, and R₂=a hydrogen atom, or
R₂=ethyl, and R₁=a hydrogen atom

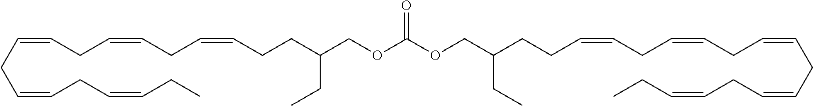

### (all-Z)-2-ethyl-5,8,11,14,17-eicosapentaen-1-ol (all-Z)-2-ethyl-5,8,11,14,17-eicosapenatenyl carbonate (86)

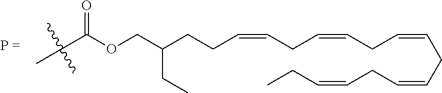

Y═C₁₈ alkenyl with 5 methylene interrupted double bonds in Z-configuration
R₁=ethyl, and R₂=a hydrogen atom, or
R₂=ethyl, and R₁=a hydrogen atom

### Methods for Preparing Compounds According to the Invention

Method (General)

All the alcohols according to the invention can be prepared from their corresponding carboxylic acids or esters under reduction conditions.

2-substituted omega-3 polyunsaturated esters or carboxylic acids can be reduced to their corresponding alcohols by using a reagent that transfers a hydride to the carbonyl compound. Examples of such reducing agents are: Lithium aluminium hydrides such as LiAlH₄, LiAlH₂(OCH₂CH₂OCH₃), LiAlH[OC(CH₃)₃]₃ or boron hydrides such as: LiBH₄, Ca(BH₄)₂. Suitable solvents include diethylether or THF are usually used in this reduction reaction under anhydrous condition.

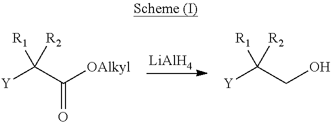

Moreover, the syntheses of 2-substituted ethyl (all-Z)-4,7,10,13,16,19-docosahexaenoate are described in patent application IB2006/001155.

### Method II: Preparation of Esters of Omega-3 Polyunsaturated Alcohols

The most general methods of synthesis of esters are by reaction of alcohols with an acid chloride or other activated carboxylic acid derivatives. Usually preparative procedures often use pyridine as a catalyst when reacting the alcohol with an acid chloride. 4-dimethyl-aminopyridine (DMAP) is also an attractive alternative as catalyst in this reaction. It is also a possibility to use a Fisher esterification procedure in where the alcohol is reacted with a carboxylic acid in the presence of an acid-catalyst.

Scheme (II) illustrates an example for preparation of pro-drugs of omega-3 polyunsaturated alcohols.

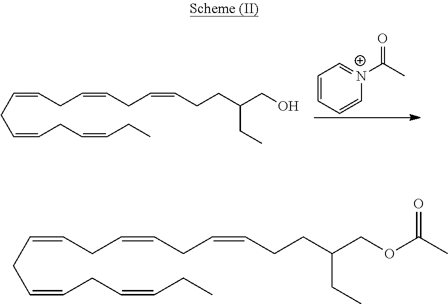

### Method III: Preparation of Phosphonates of Omega-3 Polyunsaturated Alcohols

The t-butyl protected phosphonates can be prepared by reaction of the alcohols with di-tent-butyl diisopropylphosphoramidite and hydrogen peroxide in the presence of tetrazole. Deprotection by trifluoroacetic acid yields the phosphonates (Scheme III).

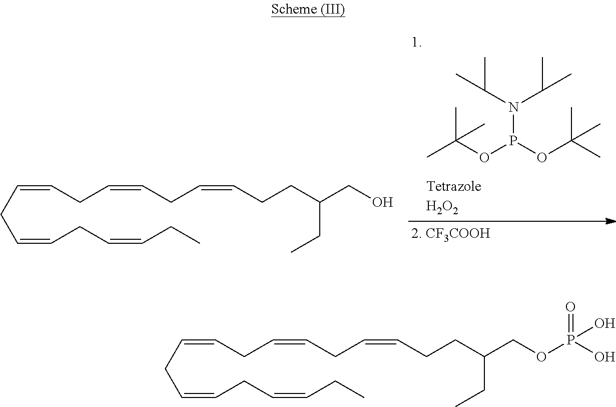

### Method IV: Preparation of Sulphonates of Omega-3 Polyunsaturated Alcohols

The sulphonates can be prepared by reaction of the alcohols with pyridine×SO₃ as shown in Scheme (IV).

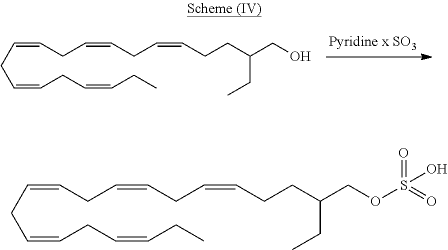

### Method V: Preparation of Omega-3 Polyunsaturated Esters of Omega-3 Polyunsaturated Alcohols

A general method involves reacting one equivalent of the polyunsaturated fatty acid with one equivalent of the polyunsaturated alcohol in the precence of EDC (1-Ethyl-3-[3-dimethylaminopropyl]carbodiimide hydrochloride), or another activator for carboxylic acids, and a base (like triethylamine or diisopropylethylamine) in an appropriate solvent. An example is shown in Scheme (V).

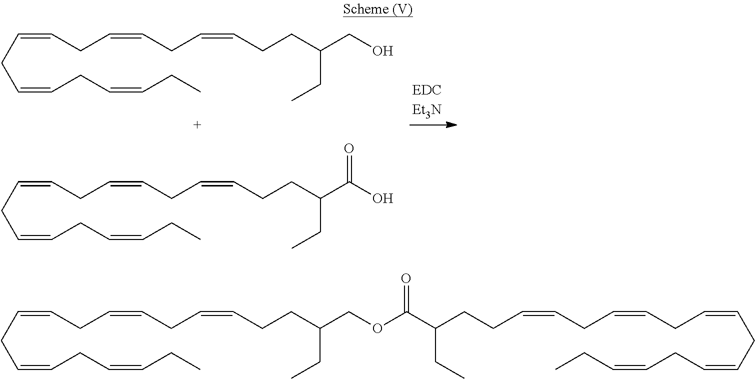

The carbonates can be prepared by reaction of the alcohol with di-t-butyl-dicarbonate (Boc-O-Boc) in the presence of a base (like DMAP) as shown in Scheme (VI).

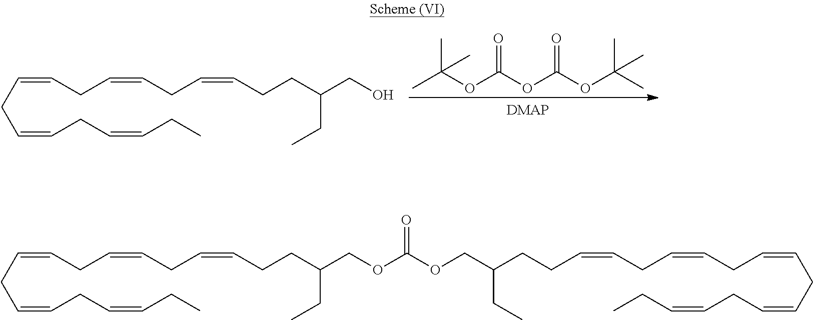

### Synthesis Protocols

### Preparation of 2-ethyl-4,7,10,13,16,19-docosahexaen-1-ol (Lipid compound 2)

### Ethyl 2-ethyl-4,7,10,13,16,19-docosahexaenoate

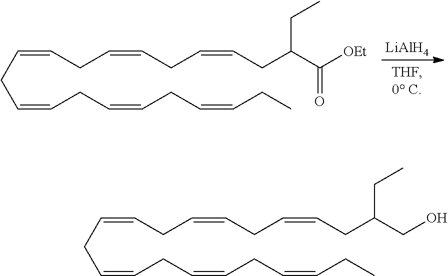

(PRB-2, 0.81 g, 2.11 mmol) in 5 mL dry THF was added to a stirred suspension of LAH (0.084 g, 2.21 mmol) in 15 mL dry THF held at 0° C. The resulting solution was stirred at 0° C. under inert atmosphere for 30 minutes, added 10% NH₄Cl (20 mL) and filtrated through a short pad of celite. The pad was washed with water (20 mL) and heptane (20 mL) and the layers were separated. The aqueous phase was extracted with heptane (20 mL) and the combined organic layer was washed with brine (20 mL) and dried (MgSO₄). Purification by flash chromatography (heptane: EtOAc 9:1) afforded 0.33 g (46%) of the title compound as a colorless oil; ¹H-NMR (200 MHz, CDCl₃): δ 0.86-0.98 (m, 6H), 1.26-1.54 (m, 3H), 1.98-2.17 (m, 4H), 2.76-2.90 (m, 10H), 3.51 (d, 2H), 5.27-5.48 (m, 12H); ¹³C-NMR (50 MHz, CDCl₃): δ 11.29, 14.18, 20.47, 23.33, 25.46, 25.54, 25.57, 25.58, 25.60, 28.41, 42.50, 65.05, 126.94, 127.78, 128.01, 128.02, 128.07, 128.11, 128.17, 128.20, 128.48, 128.99, 131.93; MS (electrospray): 365.3 [M+Na].

### (all-Z)-2-ethyl-4,7,10,13,16,19-docosahexaen-1-ol acetate

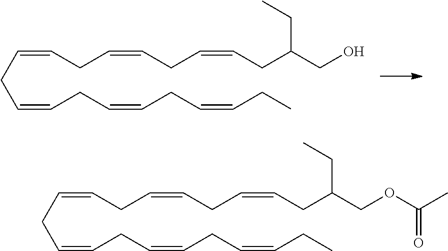

Acetyl chloride (5.64 ml, 65.6 mmol) was added drop wise to a stirred mixture of (all-Z)-2-ethyl-4,7,10,13,16,19-docosahexaen-1-ol (20.4 g, 59.6 mmol) and pyridine (46 μL, 0.6 mmol) in dry THF (200 ml) at 0° C. The mixture was allowed to slowly reach room temperature and stirred over night. The reaction was quenched with saturated NaHCO₃ (120 mL) and the resulting mixture was extracted with heptane (200 mL). The organic layer was washed with water (120 mL) and dried (Na₂SO₄). Purification by flash chromatography on silica gel eluting with heptane/EtOAc 100:2.5-95:3-95:5 afforded 18 g (79%) of the title compound as a yellow oil.

¹H-NMR (300 MHz, CDCl₃): δ 0.90 (t, 3H, J=7.4 Hz), 0.95 (t, 3H, J=7.5 Hz), 1.35 (quint., 2H, J=7.2 Hz), 1.64 (quint., 1H, J=6.4 Hz), 2.02 (s, 3H), 2.05-2.11 (m, 4H), 2.74-2.84 (m, 10H), 3.96 (d, 2H, J=5.9 Hz), 5.26-5.42 (m, 12H); ¹³C-NMR (75 MHz, CDCl₃): δ 11.2, 14.2, 20.5, 20.9, 23.6, 25.5, 25.6, 28.4, 39.3, 66.4, 127.0, 127.4, 127.8, 128.0, 128.1, 128.17, 128.19, 128.2, 128.5, 129.4, 132.0, 171.2 (4 signals hidden); MS (electrospray): 407.3 [M+Na]⁺

### (all-Z)-2-ethyl-4,7,10,13,16,19-docosahexaen-1-ol pivaloate

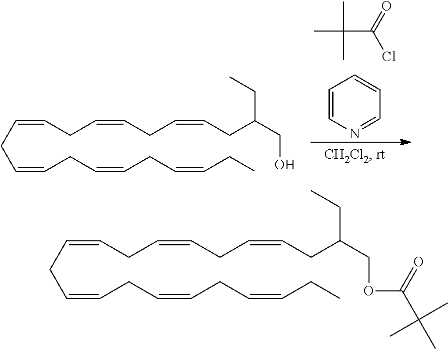

Pivaloyl chloride (71 μl, 0.58 mmol) was added to a mixture of (all-Z)-2-ethyl-4,7,10,13,16,19-docosahexaen-1-ol (200 mg, 0.59 mmol) and pyridine (0.05 ml, 0.62 mmol) in dry CH₂Cl₂ (2 ml), and the reaction mixture was stirred at room temperature under N₂-atmosphere for 42 hrs. The reaction mixture was diluted with diethyl ether (50 ml), washed with water (20 ml) and brine (20 ml), dried (Na₂SO₄) and evaporated in vacuo. Flash chromatography on silica gel eluting with heptane-heptane:EtOAc (100:1) yielded 195 mg (79%) of the title compound as a colorless liquid.

¹H NMR (200 MHz, CDCl₃) δ 0.87-0.95 (m, 6H), 1.18 (s, 9H), 1.33-1.44 (m, 2H), 1.58-1.73 (m, 1H), 1.99-2.13 (m, 4H), 2.78-2.83 (m, 10H), 3.96 (d, J=5.6 Hz, 2H), 5.23-5.48 (m, 12H); MS (electrospray); 449 [M+Na]⁺

### (all-Z)-2-ethyl-4,7,10,13,16,19-docosahexaen-1-ol hemisuccinate

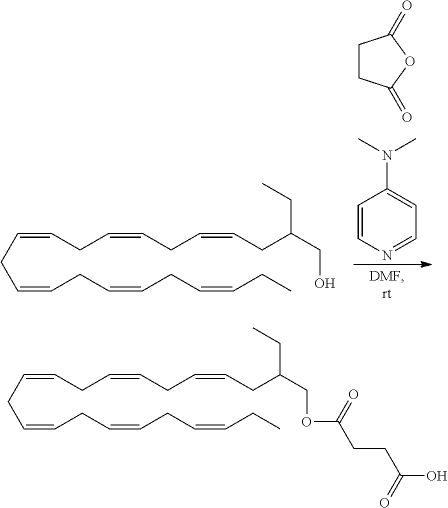

A mixture of (all-Z)-2-ethyl-4,7,10,13,16,19-docosahexaen-1-ol (200 mg, 0.59 mmol), succinic acid anhydride (65.9 mg, 0.66 mmol) and DMAP (71.6 mg, 0.59 mmol) in dry DMF (2 ml) was stirred at room temperature under N₂-atmosphere for 18 hrs. The reaction mixture was diluted with diethyl ether (50 ml), washed with 1 M HCl (20 ml) and brine (20 ml), dried (Na₂SO₄) and evaporated in vacuo. Flash chromatography on silica gel eluting with heptane:EtOAc (95:5)-(4:1)-(1:1) yielded 89 mg (34%) of the title compound as a yellow liquid.

¹H NMR (200 MHz, CDCl₃) δ 0.86-0.99 (m, 6H), 1.24-1.39 (m, 2H), 1.62-1.68 (m, 1H), 2.02-2.13 (m, 4H), 2.62-2.83 (m, 4H), 2.71-2.83 (m, 10H), 4.01 (d, J=5.8 Hz, 2H), 5.22-5.48 (m, 12H); MS (electrospray); 465 [M+Na]⁺, 441 [M−H]⁻

### (all-Z)-2-ethyl-4,7,10,13,16,19-docosahexaen-1-ol phosphonate

### Step 1: (all-Z)-2-ethyl-4,7,10,13,16,19-docosahexaen-1-ol dig-butyl phosphonate

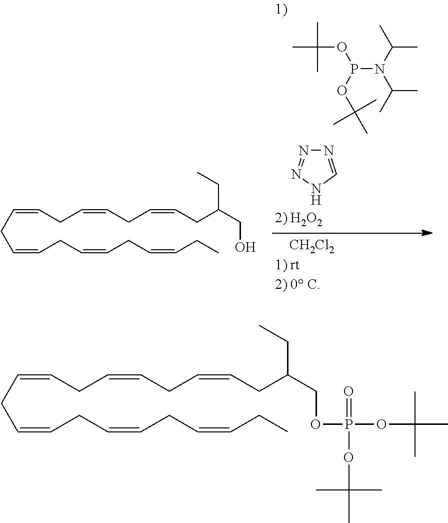

A solution of tetrazole in CH₃CN (0.45 M, 9.2 ml, 4.14 mmol) was added to a solution of (all-Z)-2-ethyl-4,7,10,13,16,19-docosahexaen-1-ol (428 mg, 1.25 mmol) and di-tert-butyl diisopropylphosphoramidite (0.635 ml, 2.01 mmol) in dry CH₂Cl₂ (30 ml). After 130 minutes of stirring at room temperature under N₂-atmosphere the mixture was cooled to 0° C. and 50% H₂O₂ (150 μl) was added. The mixture was stirred for 2 hrs at 0° C., diluted with CH₂Cl₂ (100 ml) and washed with 10% Na₂S₂O₅ (30 ml×2), water (30 ml), NaHCO₃ sat. (30 ml×2) and brine (30 ml), dried (Na₂SO₄) and evaporated in vacuo. Flash chromatography on silica gel eluting with heptane-heptane:EtOAc (95:5)-(9:1) yielded 139 mg (21%) of the title compound as a colorless liquid.

¹H NMR (200 MHz, CDCl₃) δ 0.86-0.99 (m, 6H), 1.24-1.42 (m, 2H), 1.46 (s, 18H), 1.54-1.65 (m, 1H), 1.99-2.14 (m, 4H), 2.79-2.83 (m, 10H), 3.85 (t, J=5.6 Hz, 2H), 5.23-5.5.42 (m, 12H); MS (electrospray); 557 [M+Na]⁺

### Step 2: (all-Z)-2-ethyl-4,7,10,13,16,19-docosahexaen-1-ol phosphonate

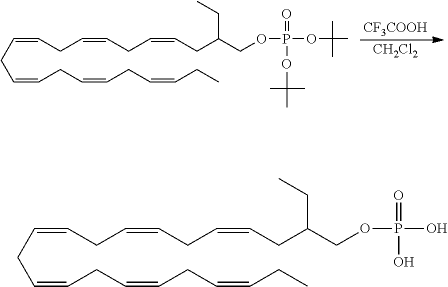

To a solution of (all-Z)-2-ethyl-4,7,10,13,16,19-docosahexaen-1-ol di-t-butyl phosphonate (133 mg, 0.25 mmol) in dry CH₂Cl₂ (25 ml) was added CF₃COOH (0.26 ml, 3.40 mmol). The mixture was stirred for 4 hrs and evaporated in vacuo. CH₂Cl₂ (20 ml) was added to the residue and the mixture was evaporated in vacuo yielding 102 mg (97%) of the title compound.

¹H NMR (200 MHz, CDCl₃) δ 0.86-0.99 (m, 6H), 1.31-1.45 (m, 2H), 1.62-1.68 (m, 1H), 2.02-2.10 (m, 4H), 2.79-2.83 (m, 10H), 3.97 (t, J=5.3 Hz, 2H), 5.23-5.48 (m, 12H), 8.91 (bs, 2H); MS (electrospray); 421 [M−H]⁻

### (all Z)-2-ethyl-4,7,10,13,16,19-docosahexaen-1-ol sulphonate

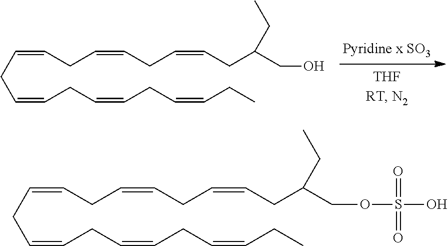

Pyridine×SO₃ (45% SO₃, 0.19 g, 1.16 mmol was added to a solution of (all-Z)-2-ethyl-4,7,10,13,16,19-docosahexaen-1-ol (0.20 g, 0.58 mmol) in dry THF (10 ml). The mixture was stirred at ambient temperature under inert atmosphere for 18 hours and portioned between 1M HCl (20 mL) and diethyl ether (20 mL). The aqueous phase was extracted with diethyl ether (20 mL), the combined organic extracts were washed with brine (20 mL), dried (Na₂SO₄) and concentrated in vacuo. The crude oil was purified by flash chromatography on a short silica column (EtOAc, then 10% MeOH in EtOAc), and this provided 0.12 g (50%) of the title compound as a pale yellow solid.

¹H-NMR (200 MHz, CDCl₃): δ 0.86 (t, 3H), 0.94 (t, 3H), 1.23-1.37 (m, 2H), 1.60-1.75 (m, 1H), 1.97-2.11 (m, 4H), 2.70-2.87 (m, 10H), 4.00 (d, 2H), 5.21-5.45 (m, 12H); MS (electrospray): 421.2 [M−H]⁻.

### (all-Z)-2-ethyl-4,7,10,13,16,19-docosahexaen-1-ol t-butyl carbonate

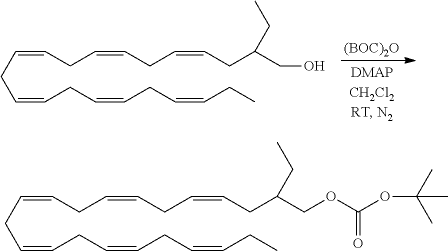

di-tert-Butyl dicarbonate (0.80 g, 3.65 mmol) was added to a solution of (all-Z)-2-ethyl-4,7,10,13,16,19-docosahexaen-1-ol 0.25 g, 0.73 mmol) and DMAP (0.089 g, 0.73 mmol) in dry CH₂Cl₂ (10 ml) under inert atmosphere. The mixture was stirred at ambient temperature for three hours. The mixture was then diluted with CH₂Cl₂ (15 mL), washed with water (2×15 mL) and brine (15 mL), dried (Na₂SO₄) and concentrated in vacuo. The crude oil was purified first by flash chromatography on silica gel (heptane:EtOAc 98:2), then by flash chromatography on reverse phase C₈ silica gel (H₂O, then H₂O:CH₃CN 50:50) to afford 0.016 g (5%) of the title compound as a colorless oil.

¹H-NMR (200 MHz, CDCl₃): δ 0.89 (t, 3H), 0.95 (t, 3H), 1.32-1.40 (m, 2H), 1.46 (s, 9H), 1.60-1.80 (m, 1H), 2.02-2.14 (m, 4H), 2.76-2.85 (m, 10H), 3.95 (d, 2H), 5.23-5.48 (m, 12H); MS (electrospray): 465.3 [M+Na].

The invention shall not be limited the shown embodiments and examples.

1-80. (canceled)

81. A compound of formula (I): 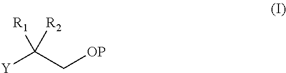
wherein R₁ and R₂ are the same or different and are chosen from a hydrogen atom, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
P represents H; 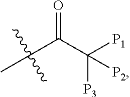
wherein P₁, P₂, and P₃ are the same or different and are chosen from a hydrogen atom, an alkyl group, and a C₁₄-C₂₂ alkenyl group, wherein the alkyl and alkenyl groups are optionally substituted with a hydroxy group; 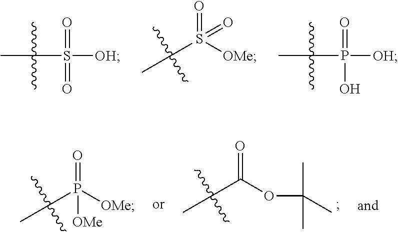
Y is a C₁₄-C₂₂ alkenyl group with at least one double bond, having E and/or Z configuration;
or any pharmaceutically acceptable complex, solvate, pro-drug, or salt thereof, with the proviso that R₁ and R₂ are not simultaneously a hydrogen atom, and when R₁ and/or R₂ is an alkenyl group, the alkenyl group is chosen from allyl, 2-butenyl, and 3-hexenyl.

82. A compound according to claim 81, wherein Y is a C₁₄-C₂₂ alkenyl with 2-6 double bonds.

83. A compound according to claim 81, wherein Y is a C₁₄-C₂₂ alkenyl with 2-6 methylene interrupted double bonds in Z configuration.

84. A compound according to claim 81, wherein Y is a C₂₀ alkenyl with 6 methylene interrupted double bonds in Z configuration.

85. A compound according to claim 81, wherein Y is a C₁₈ alkenyl with 5 methylene interrupted double bonds in Z configuration.

86. A compound according to claim 81, wherein Y is a C₁₆ alkenyl with 5 methylene interrupted double bonds in Z configuration.

87. A compound according to claim 81, wherein Y is a C₁₋₄ alkenyl with 3 methylene interrupted double bonds in Z configuration.

88. A compound according to claim 81, wherein said R₁ and R₂ are the same or different and are chosen from methyl, ethyl, n-propyl, isopropyl, n-butyl, iso-butyl, sec-butyl, and n-hexyl.

89. A compound according to claim 88, wherein said R₁ and R₂ are the same or different and are chosen from methyl, ethyl, and n-propyl.

90. A compound according to claim 81, wherein said R₁ and R₂ are the same or different and are chosen methoxy, ethoxy, propoxy, isopropoxy, sec-butoxy, phenoxy, benzyloxy, OCH₂CF₃, and OCH₂CH₂OCH₃.

91. A compound according to claim 81, wherein R₁ and R₂ are different.

92. A compound according to claim 91, in racemic form.

93. A compound according to claim 91, in the form of its R stereoisomer.

94. A compound according to claim 91, in the form of its S stereoisomer.

95. A compound according to claim 81, wherein Y is a C₁₄-C₂₂ alkenyl group with at least one double bond, having Z configuration, and having the first double bond at the third carbon-carbon bond from the omega (ω) end of the carbon chain.

96. A compound of formula (I) 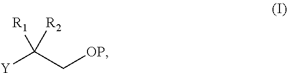
wherein R₁ and R₂ are the same or different and are chosen from a hydrogen atom, an alkyl group, and an alkoxy group;
P represents 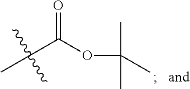
Y is chosen from a C₁₈ alkenyl group with 5 methylene interrupted double bonds in Z configuration and a C₂₀ alkenyl group with 6 methylene interrupted double bonds in Z configuration;
or any pharmaceutically acceptable complex, solvate, pro-drug, or salt thereof.

97. A compound of formula (I) 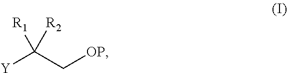
wherein R₁ and R₂ are the same or different and are chosen from a hydrogen atom, an alkyl group, and an alkoxy group;
P represents 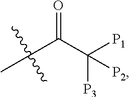
wherein P₁, P₂, and P₃ are chosen from a hydrogen atom, an alkyl group, and a C₁₄-C₂₂ alkenyl group, wherein the alkyl and alkenyl groups are optionally substituted with a hydroxy group; and
Y is chosen from a C₁₈ alkenyl group with 5 methylene interrupted double bonds in Z configuration and a C₂₀ alkenyl group with 6 methylene interrupted double bonds in Z configuration;
or any pharmaceutically acceptable complex, solvate, pro-drug, or salt thereof.

98. A compound of formula (I) 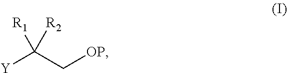
wherein R₁ and R₂ are the same or different and are chosen from a hydrogen atom, an alkyl group, and an alkoxy group;
P represents H; and
Y is chosen from a C₁₈ alkenyl group with 5 methylene interrupted double bonds in Z configuration and a C₂₀ alkenyl group with 6 methylene interrupted double bonds in Z configuration;
or any pharmaceutically acceptable complex, solvate, pro-drug, or salt thereof.

99. A compound of formula (I) 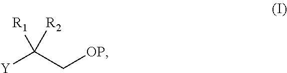
wherein R₁ and R₂ are the same or different and are chosen from a hydrogen atom, an alkyl group, and an alkoxy group;
P represents 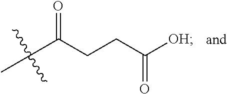
Y is chosen from a C₁₈ alkenyl group with 5 methylene interrupted double bonds in Z configuration and a C₂₀ alkenyl group with 6 methylene interrupted double bonds in Z configuration;
or any pharmaceutically acceptable complex, solvate, pro-drug, or salt thereof.

100. A compound according to claim 81 for use as a medicament or for cosmetic use as a topical preparation for use on the skin.

101. A pharmaceutical composition comprising a compound according to claim 81.

102. A pharmaceutical composition according to claim 101, further comprising a pharmaceutically acceptable carrier, excipient, or diluent, or any combination thereof.

103. A pharmaceutical composition according to claim 101, formulated for oral administration.

104. A pharmaceutical composition according to claim 103, in the form of a capsule, a sachet, a solid, or a powder.

105. A pharmaceutical composition according to claim 101, formulated to provide a daily dosage of from 1 mg to 10 g of said compound.

106. A pharmaceutical composition according to claim 105, formulated to provide a daily dosage of from 1 mg to 1 g of said compound.

107. A pharmaceutical composition according to claim 106, formulated to provide a daily dosage of from 1 mg to 500 mg of said compound.

108. A pharmaceutical composition according to claim 107, formulated to provide a daily dosage of from 50 mg to 250 mg of said compound.

109. A method for preparing a compound according to claim 81, wherein P represents 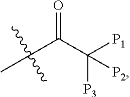
and P₁, P₂, and P₃ are each a hydrogen atom comprising:
reacting a compound according to formula (I) 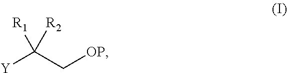
wherein P is hydrogen with acetyl chloride and pyridine in a solvent.

110. The method according to claim 109, wherein the solvent is tetrahydrofuran (THF).

111. A method for preparing a compound according to claim 81, wherein P represents 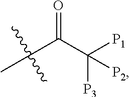
and P₁, P₂, and P₃ are each a methyl group comprising:
reacting a compound according to formula (I) 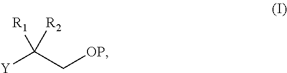
wherein P is hydrogen with pivaloyl chloride and pyridine in a solvent.

112. The method according to claim 111, wherein the solvent is methylene chloride (CH₂Cl₂).

113. A method for preparing a compound according to claim 81, wherein P represents H comprising reducing a compound of formula 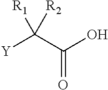
or a derivative thereof with a hydride reducing agent.

114. The method of claim 113, wherein the hydride reducing agent is chosen from LiAlH₄, LiAlH₂(OCH₂CH₂OCH₃)₂, LiAlH[OC(CH₃)₃]₃, LiBH₄, and Ca(BH₄)₂.

115. A method for preparing a compound according to claim 99, comprising reacting a compound according to formula (I) 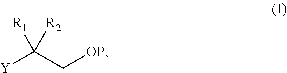
wherein P is hydrogen with succinic acid anhydride and dimethylaminopyridine (DMAP) in solvent.

116. The method according to claim 115, wherein the solvent is dimethylformamide (DMF).

117. A method for activation or modulation of at least one of the human peroxisome proliferator-activated receptor (PPAR) isoforms comprising administering to a human in need thereof a compound according to claim 81.

118. The method according to claim 117, wherein said peroxisome proliferator-activated receptor (PPAR) is PPAR α.

119. The method according to claim 117, wherein said peroxisome proliferator-activated receptor (PPAR) is peroxisome proliferator-activated receptor (PPAR) α and/or γ.

120. A method for the treatment and/or the prevention of peripheral insulin resistance and/or a diabetic condition comprising administering to a human in need thereof a compound according to claim 81.

121. A method for reduction of plasma insulin, blood glucose, and/or serum triglycerides comprising administering to a human in need thereof a compound according to claim 81.

122. A method for the treatment and/or the prevention of type 2 diabetes comprising administering to a human in need thereof a compound according to claim 81.

123. A method for the prevention and/or treatment of elevated triglyceride levels and non-HDL cholesterol levels (LDL and/or VLDL cholesterol levels) comprising administering to a human in need thereof a compound according to claim 81.

124. A method for the prevention and/or treatment of a hyperlipidemic condition comprising administering to a human in need thereof a compound according to claim 81.

125. The method according to claim 124, wherein said hyperlipidemic condition is hypertriglyceridemia (HTG).

126. A method for increasing serum HDL levels in humans comprising administering to a human in need thereof a compound according to claim 81.

127. A method for the treatment and/or the prevention of obesity or an overweight condition comprising administering to a human in need thereof a compound according to claim 81.

128. A method for reduction of body weight and/or for preventing body weight gain comprising administering to a human in need thereof a compound according to claim 81.

129. A method for the treatment and/or the prevention of a fatty liver disease comprising administering to a human in need thereof a compound according to claim 81.

130. The method according to claim 129, wherein said fatty liver disease is non-alcoholic fatty liver disease (NAFLD).

131. A method for treatment of insulin resistance, hyperlipidemia, and/or obesity or an overweight condition comprising administering to a human in need thereof a compound according to claim 81.

132. A method for the treatment and/or the prevention of an inflammatory disease or condition comprising administering to a human in need thereof a compound according to claim 81.
